(19) United States
(12) Patent Application Publication
(10) Pub. No.: US 2023/0137010 A1
(43) Pub. Date: May 4, 2023

(54) LONG-ACTING APOMORPHINE FORMULATIONS AND INJECTORS FOR THERAPEUTIC DELIVERY OF THE SAME

(71) Applicants: Scienture, Inc., Hauppauge, NY (US); INNOCORE TECHNOLOGIES B.V., Groningen (NL)

(72) Inventors: Shankar HARIHARAN, Smithtown, NY (US); Suketu SANGHVI, Kendall Park, NJ (US); Suneel GUPTA, Sunnyvale, CA (US); Rahul SURANA, Commack, NY (US); Ivan TERZIC, Groningen (NL); Albert DOORNBOS, Groningen (NL); Rob STEENDAM, Groningen (NL)

(21) Appl. No.: 17/995,888

(22) PCT Filed: Apr. 12, 2021
(86) PCT No.: PCT/US2021/026904

§ 371 Date: Oct. 10, 2022

Related U.S. Application Data
(60) Provisional application No. 63/008,369, filed on Apr. 10, 2020.

(51) Int. Cl. A61K 31/485; A61K 9/16; A61K 9/19; A61K 9/00; A61M 5/19

(52) U.S. Cl. CPC A61K 31/485; A61K 9/1611; A61K 9/1617; A61K 9/1694; A61K 9/1647; A61K 9/19; A61K 9/0019; A61M 5/19; A61M 2207/00

(57) ABSTRACT

The present disclosure is directed to formulations, devices, kits, and methods for treating or preventing motor symptoms associated with Parkinson's disease by injection of a microsphere formulation of apomorphine free base or a pharmaceutically acceptable a salt thereof, wherein injection can be from a pre-filled injector.

### TECHNICAL FIELD

According to various aspects of this disclosure, the present disclosure relates to injectors comprising pharmaceutically acceptable microsphere formulations comprising apomorphine free base or a pharmaceutically acceptable salt thereof, methods of producing the injectors and formulations, kits, and methods of treating motor symptoms associated with Parkinson's disease with the pharmaceutically acceptable formulations and injectors.

### BACKGROUND

Apomorphine is a morphine-derived non-ergoline dopamine agonist. Current formulations of apomorphine (APO-go®, Apokyn®) are known to cause redness, swelling, pain and other injection site issues. These undesirable effects occur primarily because these formulations contain apomorphine in its hydrochloride salt form, which is acidic. Therefore, there is a need for an injectable formulation of apomorphine which avoids or minimizes the undesirable side effects of the currently available immediate release acidic hydrochloride salt formulations.

### DESCRIPTION OF FIGURES

FIG. 1. A one-compartment simulation of apomorphine concentration over 168 hours at a dosing rate of 10 mg/hr and a clearance rate of 280 L/hr, with a half-life of either 30 minutes or 90 minutes.

FIG. 2. Scanning electron microscopy image of apomorphine-loaded microspheres comprising 20LP10L20-GLL40 (120B-210023, 30.2% apomorphine) using a magnification of 600×.

FIG. 3. Particle size distribution of apomorphine-loaded microspheres comprising 20LP10L20-GLL40 (120B-210023, 30.2% apomorphine).

FIG. 4. Cumulative release of apomorphine from apomorphine-loaded microspheres comprising 20LP10L20-GLL40 (120B-210023, 30.2% apomorphine).

FIGS. 5A-5C. Scanning electron microscopy images of apomorphine-loaded microspheres comprising 60LP2L20-D27 (120B-210036) (A), 20LP10L20-GLL40 (120B-210037) (B), and 5CP30C40-L40 (120B-210038) (C) using a magnification of 600×.

FIG. 6. Cumulative release of apomorphine from apomorphine-loaded microspheres comprising 60LP2L20-D27 (120B-210036), 20LP10L20-GLL40 (120B-210037) and 5CP30C40-L40 (120B-210038).

FIG. 7. Cumulative release of apomorphine from apomorphine-loaded microspheres comprising 60LP2L20-D27 (120B-210051), 20LP10L20-GLL40 (120B-210052) and 5CP30C40-L40 (120B-210053).

FIGS. 8A-8C. Scanning electron microscopy images of apomorphine-loaded microspheres comprising a 50/50% w/w blend of 10LP10L20-GLL40 and 20LP10L20-GLL40 (120B-210132, 36.5% apomorphine using a magnification of 50× (A), 200× (B) and 600× (C).

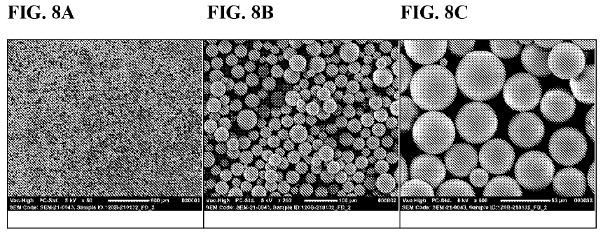

FIG. 9. Particle size distribution of apomorphine-loaded microspheres comprising a 50/50% w/w blend of 10LP10L20-GLL40 and 20LP10L20-GLL40 (120B-210132).

FIG. 10. Cumulative release of apomorphine from apomorphine-loaded microspheres comprising 20LP10L20-GLL40 (120B-210052), 10LP10L20-GLL40 (120B-210105) and a 50/50% w/w blend of 10LP10L20-GLL40 and 20LP10L20-GLL40 (120B-210132).

### BRIEF SUMMARY

### Stable Pharmaceutically Acceptable Formulation

In some aspects, the present disclosure provides a stable pharmaceutically acceptable formulation comprising a microsphere, the microsphere comprising a first biodegradable polymer and an active drug load of apomorphine or a pharmaceutically acceptable salt thereof.

In some aspects, the pharmaceutically acceptable salt is an acid addition salt of apomorphine.

In some aspects, the pharmaceutically acceptable salt is apomorphine hydrochloride.

In some aspects, the stable pharmaceutically acceptable formulation further comprises one or more antioxidants. In some aspects, the one or more antioxidants comprises sodium metabisulfite. In some aspects, the antioxidant is sodium metabisulfite. In some aspects, the one or more antioxidants comprises sodium ascorbate. In some aspects, the antioxidant is sodium ascorbate.

In some aspects, the microsphere further comprises one or more additional biodegradable polymers.

In some aspects, the first biodegradable polymer is a copolymer. In some aspects, the first biodegradable polymer is a multi-block copolymer. In some aspects, the multi-block copolymer comprises at least one hydrolysable pre-polymer (A) segment and at least one hydrolysable pre-polymer (B) segment, wherein the segments are linked by a multifunctional chain extender, and wherein the segments are randomly and non-alternatingly distributed over the polymer chain.

In some aspects, the multi-block copolymer has a T_{g} of about 37° C. or less and a Tₘ of about 110° C. to about 250° C. under physiological conditions, and wherein the pre-polymer (A) segment comprises polyethylene glycol.

In some aspects, the polyethylene glycol has a Mₙ of about 150 to about 5000 g/mol.

In some aspects, the multi-block copolymer is amorphous and has a glass transition temperature of 37° C. or less at physiological conditions.

In some aspects, the pre-polymer (A) segment and/or the pre-polymer (B) segment comprises one or more linkages selected from the group consisting of: ester linkages, carbonate linkages, anhydride linkages, ether linkages, and combinations thereof. In some aspects, the pre-polymer (A) segment comprises one or more polyether groups. In some aspects, the one or more polyether groups are selected from the group consisting of: polyethylene glycol, polyethylene glycol-polypropylene glycol, polytetramethylene ether glycol, and combinations thereof. In some aspects, said polyether group is polyethylene glycol. In some aspects, a polyether is present as an additional pre-polymer in the multi-block copolymer.

In some aspects, the pre-polymer (A) segment comprises products of a reaction of at least one cyclic monomer with at least one non-cyclic initiator selected from the group consisting of diols, dicarboxylic acids and hydroxycarboxylic acids. In some aspects, the at least one cyclic monomer is selected from the group consisting of glycolide, lactide (D and/or L), ε-caprolactone, δ-valerolactone, trimethylene carbonate, 1,4-dioxane-2-one (para-dioxanone), 1,5-dioxanone-2-one, and a cyclic anhydride. In some aspects, the at least one non-cyclic initiator is selected from the group consisting of succinic acid, glutaric acid, adipic acid, sebacic acid, lactic acid, glycolic acid, ethylene glycol, diethylene glycol, 1,4-butanediol, and 1,6-hexanediol.

In some aspects, the pre-polymer (A) segment comprises reaction products of ester forming monomers selected from diols, dicarboxylic acids, and hydroxycarboxylic acids, preferably the pre-polymer (A) segment comprises reaction products of glycolide, lactide (D and/or L), ε-caprolactone, and/or δ-valerolactone.

In some aspects, the content of pre-polymer (A) in the multi-block copolymer is from about 1% to about 90% based on total weight of the multi-block copolymer. In some aspects, the pre-polymer (A) segment has a Mₙ of about 500 g/mol or more.

In some aspects, the pre-polymer (B) segment comprises a polymer derived from hydroxyalkanoate, glycolide lactide (D and/or L), ε-caprolactone, δ-valerolactone, trimethylene carbonate, 1,4-dioxane-2-one or combinations thereof.

In some aspects, the pre-polymer (B) segment comprises poly(glycolide-co-L lactide). In some aspects, the pre-polymer (B) segment comprises poly(glycolide-co-L lactide) with a Mₙ of about 1000 g/mol or more.

In some aspects, the pre-polymer (B) segment comprises a molar amount of about 1% to about 90% of glycolide relative to combined molar amount of glycolide and L-lactide.

In some aspects, the multi-block copolymer comprises from about 10% to about 99% of the pre-polymer (B) segment relative to the total weight of the multi-block copolymer.

In some aspects, the multifunctional chain extender is a difunctional aliphatic chain extender. In some aspects, the difunctional aliphatic chain extender is a diisocyanate. In some aspects, the diisocyanate is 1,4-butane diisocyanate.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not substantially identical in composition to the second biodegradable polymer.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microsphere is about 40% w/w or less. In some aspects, the active drug load of apomorphine or a pharmaceutically acceptable salt thereof in the microsphere is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less.

In some aspects, the stable pharmaceutically acceptable formulation is substantially sterile.

In some aspects, the stable pharmaceutically acceptable formulation comprises less than 5 wt % of an apomorphine-derived impurity after sealed storage for 24 months at a temperature of 25° C. In some aspects, the stable pharmaceutically acceptable formulation comprises less than 2% wt/wt of an apomorphine-derived impurity after sealed storage for 24 months at a temperature of 25° C. In some aspects, the stable pharmaceutically acceptable formulation comprises less than 1% wt/wt of an apomorphine-derived impurity after sealed storage for 24 months at a temperature of 25° C.

In some aspects, the stable pharmaceutically acceptable formulation is substantially free of an apomorphine-derived impurity after sealed storage for 24 months at a temperature of 25° C.

In some aspects, the apomorphine-derived impurity is an apomorphine oxidation product. Applicable apomorphine oxidation products include, but are not limited to, those described in Garrido, J. M. P. J., et al. 2002. Oxidative behavior of apomorphine and its metabolites. Bioelectrochemistry. 55(1-2):113-114; and Kaul, P. N. et al. 1961. Auto-oxidation of Apomorphine. Journal of Pharmaceutical Sciences. 50(3):266-267.

In some aspects, the stable pharmaceutically acceptable formulation has a shelf life of about 14 days at 25° C. following refrigeration.

In some aspects, the stable pharmaceutically acceptable formulation has a shelf life of about 24 months at 25° C.

In some aspects, the stable pharmaceutically acceptable formulation is stored in a sterile container of about 3.0 mL capacity.

In some aspects, the stable pharmaceutically acceptable formulation is provided in an injector.

In some aspects, the injector is a disposable pen injector.

In some aspects, the stable pharmaceutically acceptable formulation is suitable for parenteral administration. In some aspects, the stable pharmaceutically acceptable formulation is suitable for subcutaneous administration. In some aspects, the stable pharmaceutically acceptable formulation is suitable for intramuscular administration.

In some aspects, the amount of apomorphine or a pharmaceutically acceptable salt thereof per dose is about 40 mg to about 200 mg.

In some aspects, the amount of apomorphine or a pharmaceutically acceptable salt thereof per dose is about 80 mg to about 100 mg.

### Method of Producing Stable Pharmaceutically Acceptable Formulation

In some aspects, the present disclosure provides a method of producing a stable pharmaceutically acceptable formulation comprising microspheres. In a further aspect, the method comprises providing a first phase comprising: a first biodegradable polymer and an active drug load of apomorphine or a pharmaceutically acceptable salt thereof. In a further aspect, the method comprises adding a second phase comprising an aqueous surfactant continuously into the first phase to form an emulsion. In a further aspect, the method comprises adding a quench solution to the emulsion to produce a volume comprising microspheres. In a further aspect, the method provides washing, filtering, and drying the microspheres to reduce solvent content.

In some aspects, the present disclosure provides a method of producing a stable pharmaceutically acceptable formulation comprising microspheres. In a further aspect, the method comprises providing a first phase comprising: a first biodegradable polymer; an active drug load of apomorphine or a pharmaceutically acceptable salt thereof, and a solvent system suitable to dissolve the polymer and apomorphine. In a further aspect, the method comprises emulsifying the first phase with a second phase, thereby forming an emulsion. In a further aspect, the second phase comprises an aqueous solution that comprises a surfactant. In a further aspect, the method comprises removing a substantial portion of the solvent system from the emulsion, thereby obtaining microspheres.

In some aspects, the method comprises collecting and drying the microspheres.

In some aspects, emulsification comprises membrane emulsification. In some aspects, the step of emulsifying the first phase with the second phase comprises membrane emulsification of the first phase into the second phase. In some aspects, the step of emulsifying the first phase with the second phase comprises membrane emulsification using a membrane through which the first phase is introduced into the second phase.

In some aspects, the step of removing the substantial portion of the solvent system from the emulsion comprises extraction of the solvent system by the aqueous solution that comprises a surfactant. In some aspects, the step of removing the substantial portion of the solvent system from the emulsion comprises extraction of a first part of the solvent system by the aqueous solution that comprises a surfactant, followed by evaporation of a second part of the solvent system.

In some aspects, the substantial portion of the solvent system is about 80% to about 100% of the solvent system in the emulsion.

In some aspects, the method further comprises washing and/or filtering the microspheres.

In some aspects, the method further comprises drying the microspheres. In some aspects, the drying of the microspheres comprises one or more of lyophilization, vacuum-drying, and freeze-vacuum drying.

In some aspects, the pharmaceutically acceptable salt is an acid addition salt of apomorphine.

In some aspects, the pharmaceutically acceptable salt is apomorphine hydrochloride.

In some aspects, the first phase comprises a solvent system in which apomorphine or a pharmaceutically acceptable salt thereof has a solubility of about 25 mg/mL or more.

In some aspects, the first phase comprises one or more solvents selected from the group consisting of: dichloromethane, ethyl acetate, chloroform, methanol, benzyl alcohol, dimethyl formamide, dimethyl sulfoxide, N-methyl pyrrolidone and dimethyl acetamide.

In some aspects, the first phase comprises dichloromethane in combination with one or more additional solvents. In some aspects, the one or more additional solvents are selected from the group consisting of: dimethyl formamide, dimethyl sulfoxide, and N-methyl pyrrolidone. In some aspects, the first phase comprises dichloromethane and dimethyl sulfoxide. In some aspects, dimethyl sulfoxide is present in an amount of about 5% to about 50% relative to the combined volume of dimethyl sulfoxide and dichloromethane in the first phase.

In some aspects, the first phase comprises about 2% to about 25% by weight of the combined mass of the first biodegradable polymer and the one or more additional biodegradable polymers.

In some aspects, the first phase comprises about 2% to about 25% by weight of apomorphine or a pharmaceutically acceptable salt thereof.

In some aspects, the first phase comprises one or more antioxidants. In some aspects, the one or more antioxidants comprises sodium metabisulfite or sodium ascorbate. In some aspects, the one or more antioxidants comprises sodium metabisulfite or sodium ascorbate in an amount from about 0.01% to about 5% w/v relative to the first phase.

In some aspects, the second phase comprises polyvinyl alcohol.

In some aspects, the second phase comprises sodium chloride.

In some aspects, the second phase comprises one or more antioxidants.

In some aspects, the one or more antioxidants comprises sodium metabisulfite. In some aspects, the antioxidant is sodium metabisulfite. In some aspects, the one or more antioxidants comprises sodium metabisulfite in an amount of about 0.1% to about 1% w/v in the second phase. In some aspects, the one or more antioxidants comprises sodium metabisulfite in an amount of about 0.15% w/v in the second phase.

In some aspects, the one or more antioxidants comprises sodium ascorbate. In some aspects, the antioxidant is sodium ascorbate. In some aspects, the one or more antioxidants comprises sodium ascorbate in an amount of about 0.1% to about 1% w/v in the second phase. In some aspects, the one or more antioxidants comprises sodium ascorbate in an amount of about 0.15% w/v in the second phase.

In some aspects, the first biodegradable polymer is selected from the group consisting of a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the aqueous surfactant comprises one or more of a cationic surfactant, an anionic surfactant, or a non-ionic surfactant.

In some aspects, the second phase further comprises one or more of: a buffer solution, one or more agents for adjusting the viscosity of the aqueous surfactant, and an agent for adjusting the ionic strength of the solution.

In some aspects, the first phase is stirred prior to and/or during the addition of the second phase.

In some aspects, the emulsion is stirred prior to and/or during the addition of the quench solution.

In some aspects, the volume comprising a microsphere is stirred prior to and/or during any of the steps of washing, filtering and drying the microsphere.

In some aspects, the first biodegradable polymer is substantially enclosed by a second biodegradable polymer.

In some aspects, the first biodegradable polymer is not identical in composition to the second biodegradable polymer.

In some aspects, the first phase is prepared by mixing a solution comprising the first biodegradable polymer with a solution comprising the apomorphine or a pharmaceutically acceptable salt thereof.

In some aspects, the first biodegradable polymer and/or the second biodegradable polymer is dissolved in a solvent highly or fully miscible with water selected from the group consisting of: dimethyl sulfoxide, N-methyl-2-pyrrolidone, tetrahydrofuran, tetraglycol, acetone, an acetone/methyl ethyl ketone mixture, an acetone/methyl acetate mixture, a tetrahydrofuran/ethyl acetate mixture, and a tetrahydrofuran/ethyl formate mixture.

In some aspects, the solvent highly or fully miscible with water is an acetone/methyl ethyl ketone mixture.

In some aspects, the acetone/methyl ethyl ketone mixture comprises about 70% acetone and about 30% methyl ethyl ketone, by volume.

In some aspects, the first biodegradable polymer and/or the second biodegradable polymer is dissolved in a solvent having limited water solubility selected from the group consisting of: ethyl acetate, methyl acetate, ethyl formate, propyl formate, isopropyl formate, methyl ethyl ketone, and a mixture of two or more thereof.

### Pre-Filled Injector

In some aspects, the present disclosure provides a pre-filled injector, the pre-filled injector comprising a stable pharmaceutically acceptable formulation. In a further aspect, the formulation comprises microspheres. In a further aspect, the microspheres comprise a first biodegradable polymer and an active drug load of apomorphine or a pharmaceutically acceptable salt thereof.

In some aspects, the pharmaceutically acceptable salt is an acid addition salt of apomorphine.

In some aspects, the pharmaceutically acceptable salt is apomorphine hydrochloride.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not identical in composition to the second biodegradable polymer.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microspheres is about 40% w/w or less. In some aspects, the active drug load of apomorphine or a pharmaceutically acceptable salt thereof in the microspheres is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less.

In some aspects, the stable pharmaceutical formulation is substantially sterile.

In some aspects, the stable pharmaceutical formulation has a shelf life of about 14 days at 25° C. following refrigeration.

In some aspects, the stable pharmaceutical formulation has a shelf life of about 24 months at 25° C.

In some aspects, the pre-filled injector is a pen injector or an autoinjector.

In some aspects, the pre-filled injector is disposable.

In some aspects, the pre-filled injector is a disposable pen injector.

In some aspects, the pre-filled injector comprises a cartridge comprising the stable pharmaceutically acceptable formulation.

In some aspects, the cartridge is a dual chamber cartridge.

In some aspects, the dual chamber cartridge comprises, in a first chamber, a first volume comprising microspheres, the microspheres comprising an active drug load of apomorphine or a pharmaceutically acceptable salt thereof. In some aspects, the dual chamber cartridge comprises, in a second chamber, a second volume comprising a dilution medium.

In some aspects, the first volume and the second volume can be combined to produce a pharmaceutically acceptable formulation disclosed herein.

In some aspects, the pre-filled injector is configurable to combine the first volume and the second volume prior to injection. In some aspects, the pre-filled injector is configurable to combine the first volume and the second volume during injection.

In some aspects, the pre-filled injector is configurable to dispense the first volume and the second volume in about equal amounts by volume. In some aspects, the pre-filled injector is configurable to dispense the first volume and the second volume in amounts that are not equal by volume.

In some aspects, the first volume has a shelf life of about 7 to about 30 days at 25° C. following refrigeration. In some aspects, the first volume has a shelf life of about 7 days at 25° C. following refrigeration. In some aspects, the first volume has a shelf life of about 14 days at 25° C. following refrigeration. In some aspects, the first volume has a shelf life of about 21 days at 25° C. following refrigeration.

In some aspects, the first volume has a shelf life of about 1, to about 24 months at 25° C. In some aspects, the first volume has a shelf life of about 3 months at 25° C. In some aspects, the first volume has a shelf life of about 6 months at 25° C. In some aspects, the first volume has a shelf life of about 12 months at 25° C.

In some aspects, the second volume has a shelf life of about 7 to about 30 days at 25° C. following refrigeration. In some aspects, the second volume has a shelf life of about 7 days at 25° C. following refrigeration. In some aspects, the second volume has a shelf life of about 14 days at 25° C. following refrigeration. In some aspects, the second volume has a shelf life of about 21 days at 25° C. following refrigeration.

In some aspects, the second volume has a shelf life of about 1, to about 24 months at 25° C. In some aspects, the second volume has a shelf life of about 3 months at 25° C. In some aspects, the second volume has a shelf life of about 6 months at 25° C. In some aspects, the second volume has a shelf life of about 12 months at 25° C.

In some aspects, the pre-filled injector comprises a 18 G to 30 G needle. In some aspects, the pre-filled injector comprises a 21 G needle.

In some aspects, the amount of the stable pharmaceutically acceptable formulation dispensed in a single injection contains about 40 mg to about 200 mg of apomorphine or a pharmaceutically acceptable salt thereof.

In some aspects, the amount of the stable pharmaceutical formulation dispensed in a single injection contains about 80 mg to about 100 mg of apomorphine or a pharmaceutically acceptable salt thereof.

In some aspects, the amount of the stable pharmaceutical formulation dispensed in a single injection contains about 90 mg of apomorphine or a pharmaceutically acceptable salt thereof.

### Method of Manufacturing Pre-Filled Injector

In some aspects, the present disclosure provides a method of manufacturing a pre-filled injector comprising a stable pharmaceutically acceptable formulation. In a further aspect, the method comprises preparing a stable pharmaceutically acceptable formulation. In a further aspect, the formulation comprises microspheres. In a further aspect, the microspheres comprise a first biodegradable polymer and an active drug load of apomorphine or a pharmaceutically acceptable salt thereof. In a further aspect, the method comprises loading a sterile cartridge with the stable pharmaceutically acceptable formulation. In a further aspect, the method comprises attaching the sterile cartridge operably to an injector.

In some aspects, the pharmaceutically acceptable salt is an acid addition salt of apomorphine.

In some aspects, the pharmaceutically acceptable salt is apomorphine hydrochloride.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not identical in composition to the second biodegradable polymer.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microspheres is about 40% w/w or less. In some aspects, the active drug load of apomorphine or a pharmaceutically acceptable salt thereof in the microspheres is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less.

In some aspects, the stable pharmaceutical formulation is substantially sterile.

In some aspects, the stable pharmaceutical formulation has a shelf life of about 14 days at 25° C. following refrigeration.

In some aspects, the stable pharmaceutical formulation has a shelf life of about 24 months at 25° C.

In some aspects, the pre-filled injector is a pen injector or an autoinjector.

In some aspects, the pre-filled injector is disposable.

In some aspects, the pre-filled injector is a disposable pen injector.

In some aspects, the pre-filled injector comprises a cartridge comprising the stable pharmaceutically acceptable formulation.

In some aspects, the cartridge is a dual chamber cartridge.

In some aspects, the dual chamber cartridge comprises, in a first chamber, a first volume comprising microspheres, the microspheres comprising an active drug load of apomorphine or a pharmaceutically acceptable salt thereof. In some aspects, the dual chamber cartridge comprises, in a second chamber, a second volume comprising a dilution medium.

In some aspects, the first volume and the second volume can be combined to produce a pharmaceutically acceptable formulation disclosed herein.

In some aspects, the pre-filled injector is configurable to combine the first volume and the second volume prior to injection. In some aspects, the pre-filled injector is configurable to combine the first volume and the second volume during injection.

In some aspects, the pre-filled injector is configurable to dispense the first volume and the second volume in about equal amounts by volume. In some aspects, the pre-filled injector is configurable to dispense the first volume and the second volume in amounts that are not equal by volume.

In some aspects, the pre-filled injector comprises a 18 G to 30 G needle. In some aspects, the pre-filled injector comprises a 21 G needle.

In some aspects, the amount of the stable pharmaceutically acceptable formulation dispensed in a single injection contains about 40 mg to about 200 mg of apomorphine or a pharmaceutically acceptable salt thereof.

In some aspects, the amount of the stable pharmaceutically acceptable formulation dispensed in a single injection contains about 80 mg to about 100 mg of apomorphine or a pharmaceutically acceptable salt thereof.

In some aspects, the amount of the stable pharmaceutically acceptable formulation dispensed in a single injection contains about 90 mg of apomorphine or a pharmaceutically acceptable salt thereof.

### Method of Treatment

In some aspects, the present disclosure provides a method of treating motor symptoms associated with Parkinson's disease, the method comprising administering to a subject in need thereof a stable pharmaceutically acceptable formulation. In some aspects, the formulation comprises microspheres. In some aspects, the microspheres comprise a first biodegradable polymer and an active drug load of apomorphine or a pharmaceutically acceptable salt thereof.

In some aspects, the pharmaceutically acceptable salt is an acid addition salt of apomorphine.

In some aspects, the pharmaceutically acceptable salt is apomorphine hydrochloride.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w.

In some aspects, the amount of the stable pharmaceutically acceptable formulation administered to the subject contains an amount of apomorphine or a pharmaceutically acceptable salt thereof of about 40 mg to about 200 mg.

In some aspects, the amount of the stable pharmaceutically acceptable formulation administered to the subject contains an amount of apomorphine or a pharmaceutically acceptable salt thereof of about 80 mg to about 100 mg.

In some aspects, the amount of the stable pharmaceutically acceptable formulation administered to the subject contains an amount of apomorphine or a pharmaceutically acceptable salt thereof of about 90 mg.

In some aspects, the stable pharmaceutically acceptable formulation is administered with an injector.

In some aspects, the stable pharmaceutically acceptable formulation is administered parenterally.

In some aspects, the stable pharmaceutically acceptable formulation is administered subcutaneously or intramuscularly.

In some aspects, administration of the stable pharmaceutically acceptable formulation effects a therapeutically effective concentration of apomorphine for about 3 to about 28 days following an initial burst of apomorphine in the plasma. In some aspects, administration of the stable pharmaceutically acceptable formulation effects a therapeutically effective concentration of apomorphine for about 2 to about 14 days following an initial burst of apomorphine in the plasma. In some aspects, the therapeutically effective concentration of apomorphine is about 10 μg/L to about 200 μg/L.

In some aspects, administration of the stable pharmaceutically acceptable formulation effects a steady-state plasma concentration of apomorphine following an initial burst of apomorphine in the plasma.

In some aspects, the steady-state plasma concentration is about 35 μg/L.

In some aspects, the steady-state plasma concentration is maintained for about 7 days.

In some aspects, the initial burst of apomorphine is about 20% to about 80% of the total dose of apomorphine or a pharmaceutically acceptable salt thereof. In some aspects, the burst of apomorphine is about 20% to about 80% of the total dose of apomorphine or a pharmaceutically acceptable salt thereof in 24 hours. In some aspects, the burst of apomorphine is about 40% of the total dose of apomorphine or a pharmaceutically acceptable salt thereof in 24 hours. In some aspects, the initial burst of apomorphine is about 5% of the total dose of apomorphine or a pharmaceutically acceptable salt thereof. In some aspects, the initial burst of apomorphine is about 50% of the total dose of apomorphine or a pharmaceutically acceptable salt thereof.

In some aspects, the initial burst of apomorphine is completed about 0.5 to about 24 hours after injection.

In some aspects, the stable pharmaceutically acceptable formulation is administered twice per week. In some aspects, the stable pharmaceutically acceptable formulation is administered once per week. In some aspects, the stable pharmaceutically acceptable formulation is administered once every two weeks. In some aspects, the stable pharmaceutically acceptable formulation is administered once every three weeks. In some aspects, the stable pharmaceutically acceptable formulation is administered once every 28 days. In some aspects, the stable pharmaceutically acceptable formulation is administered once per month.

In some aspects, administration of the stable pharmaceutically acceptable formulation results in less hypotension relative to administration with immediate release subcutaneous apomorphine hydrochloride injection.

In some aspects, administration of the stable pharmaceutically acceptable formulation results in less nausea relative to administration with immediate release subcutaneous apomorphine hydrochloride injection.

In some aspects, administration of the stable pharmaceutically acceptable formulation results in less adverse injection site reactions relative to administration with immediate release subcutaneous apomorphine hydrochloride injection.

In some aspects, the subject in need suffers from Parkinson's disease.

In some aspects, administration of the stable pharmaceutically acceptable formulation is effective in reducing the occurrence, duration, or severity of intermittent episodes of muscle stiffness and/or loss of muscle control associated with Parkinson's disease.

In some aspects, administration of the stable pharmaceutically acceptable formulation is effective at minimizing hypotension or nausea, or reducing hypotension or nausea by at least 50% when compared to parenteral administration of immediate release apomorphine hydrochloride.

In some aspects, injection site reactions are substantially reduced relative to injection site reactions characteristic of injection with immediate release apomorphine hydrochloride.

### Long-Acting Dosage Form

One method of producing a long-acting dosage form of a drug is to incorporate the drug into a polymeric matrix that releases the drug slowly over time. Examples of such methods and compositions are described in published international applications WO 2013/015685 A1, WO 2005/068533 A1, and WO 2020/071912 A1. These references are incorporated in their entirety herein for all that they disclose. It will be understood by a person of ordinary skill in the relevant art that the various polymeric compositions and methods of preparing same described in the incorporated references can be used or modified to produce the long-acting apomorphine compositions described herein. In particular, any compatible polymers described therein are within the scope of use in a long-acting apomorphine composition of the present invention.

In some aspects, the present disclosure provides a long-acting dosage form comprising microspheres, the microspheres comprising a first biodegradable polymer and about 40 mg to about 200 mg of an active drug load of apomorphine or a pharmaceutically acceptable salt thereof. In some aspects, administration of a single dose of the long-acting dosage form to a subject results in at least one of the pharmacokinetic parameters selected from the group consisting of: (a) a steady state plasma profile of apomorphine from day 1 to day 7 following administration exhibiting a mean Cₘₐₓ value no greater than the steady state plasma level of apomorphine provided by 2 mg of immediate release subcutaneous injection of apomorphine hydrochloride; (b) an apomorphine elimination half-life of about 40 minutes to about 60 minutes; and (c) a zero-order or a first-order release profile of apomorphine or a pharmaceutically acceptable salt thereof.

In some aspects, administration of a single dose of the long-acting dosage form to a subject results in a zero-order release profile. In some aspects, administration of a single dose of the long-acting dosage form to a subject results in a first-order release profile.

In some aspects, the release profile corresponds to about 1% to about 50% release of the total administered dose of apomorphine or a pharmaceutically acceptable salt thereof per day. In some aspects, the release profile corresponds to about 1% to about 25% release of the total administered dose of apomorphine or a pharmaceutically acceptable salt thereof per day. In some aspects, the release profile corresponds to about 3% to about 15% release of the total administered dose of apomorphine or a pharmaceutically acceptable salt thereof per day. In some aspects, the release profile corresponds to about 12% to about 14% release of the total administered dose of apomorphine or a pharmaceutically acceptable salt thereof per day.

In some aspects, the pharmaceutically acceptable salt is an acid addition salt of apomorphine.

In some aspects, the pharmaceutically acceptable salt is apomorphine hydrochloride.

In some aspects, the long-acting dosage form comprises a plurality of microspheres comprising apomorphine or a pharmaceutically acceptable salt thereof, wherein the plurality of microspheres is characterized by a median particle size (“D50”) of about 20 μm to about 80 μm. In some aspects, the plurality of microspheres is characterized by a D50 of about 50 μm. In some aspects, the plurality of microspheres is characterized by a D50 of about 70 μm.

In some aspects, the plurality of microspheres is characterized by a D50 having a coefficient of variance (“CV”) less than 70%.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w.

In some aspects, the amount of the stable pharmaceutically acceptable formulation administered to the subject contains an amount of apomorphine or a pharmaceutically acceptable salt thereof of about 80 mg to about 100 mg.

In some aspects, the amount of the stable pharmaceutically acceptable formulation administered to the subject contains an amount of apomorphine or a pharmaceutically acceptable salt thereof of about 90 mg.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not identical in composition to the second biodegradable polymer.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microspheres is about 40% w/w or less. In some aspects, the active drug load of apomorphine or a pharmaceutically acceptable salt thereof in the microspheres is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less.

In some aspects, the stable pharmaceutical formulation is substantially sterile.

In some aspects, the stable pharmaceutical formulation has a shelf life of about 14 days at 25° C. following refrigeration.

In some aspects, the stable pharmaceutical formulation has a shelf life of about 24 months at 25° C.

In some aspects, the stable pharmaceutically acceptable formulation is administered from an injector.

In some aspects, the injector is a pen injector or an autoinjector.

In some aspects, wherein the injector is disposable.

In some aspects, the injector is a disposable pen injector.

In some aspects, the injector comprises a cartridge comprising the stable pharmaceutically acceptable formulation.

In some aspects, the cartridge is a dual chamber cartridge.

In some aspects, the dual chamber cartridge comprises, in a first chamber, a first volume comprising a microsphere, the microsphere comprising an active drug load of apomorphine or a pharmaceutically acceptable salt thereof. In some aspects, the dual chamber cartridge comprises, in a second chamber, a second volume comprising a dilution medium.

In some aspects, the first volume and the second volume can be combined to produce a pharmaceutically acceptable formulation disclosed herein.

In some aspects, the pre-filled injector is configurable to combine the first volume and the second volume prior to injection. In some aspects, the pre-filled injector is configurable to combine the first volume and the second volume during injection.

In some aspects, the pre-filled injector is configurable to dispense the first volume and the second volume in about equal amounts by volume. In some aspects, the pre-filled injector is configurable to dispense the first volume and the second volume in amounts that are not equal by volume.

In some aspects, the pre-filled injector comprises a 18 G to 30 G needle. In some aspects, the injector comprises a 21 G needle.

In some aspects, the injector is a pre-filled injector comprising the stable pharmaceutically acceptable formulation.

In some aspects, the stable pharmaceutically acceptable formulation is administered parenterally.

In some aspects, the stable pharmaceutically acceptable formulation is administered subcutaneously or intramuscularly.

In some aspects, administration of the stable pharmaceutically acceptable formulation effects a therapeutically effective concentration of apomorphine for about 3 to about 28 days following an initial burst of apomorphine in the plasma. In some aspects, administration of the stable pharmaceutically acceptable formulation effects a therapeutically effective concentration of apomorphine for about 2 to about 14 days following an initial burst of apomorphine in the plasma. In some aspects, the therapeutically effective concentration of apomorphine is about 10 μg/L to about 200 μg/L.

In some aspects, administration of the stable pharmaceutically acceptable formulation effects a steady-state plasma concentration of apomorphine following an initial burst of apomorphine in the plasma.

In some aspects, the steady-state plasma concentration is about 35 μg/L.

In some aspects, the steady-state plasma concentration is maintained for about 7 days.

In some aspects, the initial burst of apomorphine is about 20% to about 80% of the total dose of apomorphine or a pharmaceutically acceptable salt thereof. In some aspects, the burst of apomorphine is about 20% to about 80% of the total dose of apomorphine or a pharmaceutically acceptable salt thereof in 24 hours. In some aspects, the burst of apomorphine is about 40% of the total dose of apomorphine or a pharmaceutically acceptable salt thereof in 24 hours. In some aspects, the initial burst of apomorphine is about 5% of the total dose of apomorphine or a pharmaceutically acceptable salt thereof. In some aspects, the initial burst of apomorphine is about 50% of the total dose of apomorphine or a pharmaceutically acceptable salt thereof.

In some aspects, following the initial burst of apomorphine or a pharmaceutically acceptable salt thereof, there is an extended release of apomorphine for about 7 days. In some aspects, the extended release corresponds to a release of about 5% to about 25% release of the total administered dose of apomorphine or a pharmaceutically acceptable salt thereof per day. In some aspects, the extended release corresponds to a release of about 10% to about 15% release of the total administered dose of apomorphine or a pharmaceutically acceptable salt thereof per day.

In some aspects, the initial burst of apomorphine is completed about 0.5 to about 24 hours after injection.

In some aspects, following the initial burst of apomorphine or a pharmaceutically acceptable salt thereof, there is a zero-order or first-order release profile. In some aspects, there is a zero-order release profile. In some aspects, there is a first-order release profile.

In some aspects, the zero-order or first-order release profile persists for about 2, about 3, about 4, about 5, about 6, about 7, about 8, about 9, about 10, about 11, about 12, about 13, or about 14 days.

In some aspects, the zero-order release profile corresponds to a release of about 12% to about 14% release of the total administered dose of apomorphine or a pharmaceutically acceptable salt thereof per day.

In some aspects, the zero-order release profile corresponds to a release of about 5% to about 14% release of the total administered dose of apomorphine or a pharmaceutically acceptable salt thereof per day.

In some aspects, the elimination half-life of apomorphine is about 40 minutes to about 60 minutes.

In some aspects, the steady state plasma profile of apomorphine from day 1 to day 7 following administration exhibits a mean Cₘₐₓ value no greater than the steady state plasma level of apomorphine provided by 2 mg of immediate release subcutaneous injection of apomorphine hydrochloride.

In some aspects, the bioavailability of apomorphine or a pharmaceutically acceptable salt thereof is substantially the same under fed and fasting conditions.

In some aspects, administration of the stable pharmaceutically acceptable formulation results in less hypotension relative to administration with immediate release subcutaneous apomorphine hydrochloride injection.

In some aspects, administration of the stable pharmaceutically acceptable formulation results in less nausea relative to administration with immediate release subcutaneous apomorphine hydrochloride injection.

In some aspects, administration of the stable pharmaceutically acceptable formulation results in less adverse injection site reactions relative to administration with immediate release subcutaneous apomorphine hydrochloride injection.

### Kit

In some aspects, the present disclosure provides a kit. In a further aspect, the kit comprises a first vial comprising a concentrated form of the stable pharmaceutically acceptable formulations described herein or one of the long-acting dosage forms described herein. In a further aspect, the kit comprises a second vial comprising a pharmaceutically acceptable diluent. In a further aspect, the kit comprises a first syringe suitable for withdrawing the pharmaceutically acceptable diluent from the second vial. In a further aspect, the kit comprises an adapter that can operably attach to the first syringe and is suitable for dispensing the pharmaceutically acceptable diluent into the first vial. In a further aspect, the kit comprises a second syringe suitable for withdrawing a liquid from the second vial and for injecting the liquid into a subject. In a further aspect, the kit comprises instructions for diluting the concentrated form and for administering the stable pharmaceutically acceptable formulation or the long-acting dosage form to a patient in need thereof.

### DETAILED DESCRIPTION

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

As used herein, the terms “a microsphere” or “the microsphere” include multiple microspheres (e.g., a plurality of microspheres), unless the context clearly dictates otherwise. For example, a pharmaceutical composition comprising “a microsphere” should be interpreted to also encompass embodiments wherein the pharmaceutical composition comprises microspheres, or a plurality of microspheres. In a further example, a method comprising a step of “drying the microsphere” should be interpreted to also encompass embodiments wherein microspheres, or a plurality of microspheres, are dried.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” refers to the relatively non-toxic, inorganic and organic acid addition salts of apomorphine. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of apomorphine include the conventional nontoxic salts or quaternary ammonium salts of apomorphine, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. The term “excipient” refers to any substance, not itself a therapeutic agent, which can be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

The term “effective amount” or “pharmaceutically effective amount” or “therapeutically effective amount” as used herein refers to the amount or quantity of a drug or pharmaceutically active substance which is sufficient to elicit the required or desired therapeutic response, or in other words, the amount which is sufficient to elicit an appreciable biological response when administered to a patient.

The term “unit dosage form” or “unit dose composition” as used herein refers to a device containing a quantity of the therapeutic compound, said quantity being such that one or more predetermined units can be provided as a single therapeutic administration.

The term “Cₘₐₓ” as used herein refers to the maximum plasma concentration of a drug after administration of the drug.

The term “Tₘₐₓ” as used herein refers to the time required to reach the maximal plasma concentration Cₘₐₓ after administration of a drug.

The term “AUC” as used herein refers to the area under the curve of a plot of plasma concentration versus time following administration of a drug.

The term “AUC₀₋ₜ” as used herein refers to the area under the drug concentration-time curve from time zero to the time of the last measurable concentration (Ct).

The term “AUC_{0-∞}” as used herein refers to the area under the drug concentration-time curve from time zero to infinity.

The term “steady-state” as used herein means that the amount of the drug reaching the system is approximately the same as the amount of the drug leaving the system. Thus, at “steady-state,” the patient's body eliminates the drug at approximately the same rate that the drug becomes available to the patient's system through absorption into the blood stream.

The term “mean” refers to an average value in a patient population. For example, a “mean Cₘₐₓ” refers to an average of the maximum plasma concentrations of a drug in a patient population.

As used herein, the term “administration” refers to the administration of a composition (e.g., a compound or a preparation that includes a compound as described herein) to a subject or system. Administration to an animal subject (e.g., to a human) can be by any appropriate route, such as one described herein.

The term “treating” or “treatment” as used herein refers to the administration of a composition to a subject for therapeutic purposes.

The term “serum concentration” generally refers to the amount of a drug or other compound in the circulation, both bound to proteins and unbound, the latter of which generally corresponds to the therapeutically active fraction.

The term “bioavailability” generally refers to the rate and extent to which the active ingredient is absorbed from a drug product and becomes available at the site of action.

“Bioequivalence” is a term in pharmacokinetics generally used to assess the expected in vivo biological equivalence of two proprietary preparations of a drug. Two pharmaceutical products are bioequivalent if they are pharmaceutically equivalent and their bioavailabilities (rate and extent of availability) after administration in the same molar dose are similar to such a degree that their effects, with respect to both efficacy and safety, can be expected to be essentially the same.

The term “injector” as used herein refers to an apparatus wherein an individual can administer a formulation, such as a pharmaceutical formulation, to oneself. In some aspects, the injector delivers a single dose. In some aspects, the injector is adjustable to deliver various volumes of the apomorphine free base or pharmaceutically acceptable salt thereof. In some aspects, multiple injections can be dispensed from the same injector. In other aspects, part or all of the injector is disposable and/or reusable. In some aspects, part or all of the injector is opaque, and in further specific embodiments at least one part of the injector that is opaque is the part that houses the pharmaceutical formulation. An injector can be supplied separately from the pharmaceutical formulations, in alternative aspects. The injector may comprise an exchangeable vessel for replacing the pharmaceutical formulation, such as an insert, cartridge, vial, and so forth. Such an exchangeable vessel can be glass or plastic, for example. It will be understood by the skilled artisan that the injector can be an autoinjector, a pen injector, a needle-less injector, or any other injection device suitable for the delivery of a pharmaceutical formulation.

In a further aspect, apomorphine formulations can be administered via a parenteral route. As used herein, the term “parenteral” includes routes that bypass the alimentary tract. Specifically, the pharmaceutical compositions disclosed herein can be administered for example, but not limited to intravenously, intradermally, intramuscularly, intraarterially, intrathecally, subcutaneous, or intraperitoneally.

As used herein, the term “microsphere” refers to a polymeric matrix of a diameter suitable for parenteral injection. For example, a microsphere of the present disclosure can be of a diameter of about 1 μm to about 500 μm, about 5 μm to about 200 μm, about 10 μm to about 100 μm, about 20 μm to about 70 μm, about 10 nm to about 10 μm, about 20 nm to about 1 μm, about 100 nm to about 1 μm, or about 250 nm to about 750 nm. In some aspects, a microsphere of the present disclosure can be of a diameter of about 50 μm. Microspheres of the present disclosure are suitable for embedding an API within the polymeric matrix.

The term “substantially sterile” means substantially free from living microorganisms. For example, if a sterilization process removes all but about 0.001%, 0.01%, or 0.1% of the contaminants, it can be considered, in some situations, “substantially sterile”.

The term “Water for Injection” (“WFI”) refers to water that meets the U.S.P. requirements (or foreign equivalent) for “Water for Injection.” These requirements include bacterial endotoxins of not more than 0.25 U.S.P. EU per mL, total organic carbon (TOC) content of <500 parts per billion (ppb), and conductivity of 1.3 S/cm. Water for Injection also includes compendial and non-compendial water classifications that meet the requirements of U.S.P. Water for Injection. Examples include water labeled or marketed as “Low Endotoxin U.S.P. Purified Water” and “WFI Quality Water.”

The term “terminal elimination half-life” refers to the time required for half the quantity of an administered substance deposited in a living organism to be metabolized or eliminated by normal biological processes.

As used herein, “API” or “Active Pharmaceutical Ingredient” refer to apomorphine free base or a pharmaceutically acceptable salt of apomorphine.

As used herein, the term “active drug load” refers to an amount of API which is embedded into a microsphere composition. Active drug load can be expressed as the proportion by weight of API relative to the mass of the microsphere composition containing API. For example, “an active drug load of about 10% w/w apomorphine free base” refers to an amount of apomorphine free base at a proportion by weight of about 10% relative to the total weight of both the microsphere composition and the apomorphine free base contained within the microsphere composition.

As used herein, the term “long-acting” refers to the duration of action of a composition of API as disclosed and claimed herein. More specifically, the term “long-acting” refers to the period of time after which a desired blood plasma apomorphine level is maintained after a certain dose of a formulation disclosed herein has been administered. In one non-limiting example, the formulations of the present invention provide effective plasma apomorphine levels over a period of more than about 1 day to about 10 days after a single dose to a subject in need thereof. In alternative non-limiting examples, the formulations disclosed herein can provide effective plasma apomorphine levels after a single dose to a subject in need thereof over a period of more than about 1 day, about 2 days, about 3 days, about 4 days, about 5 days, about 6 days, about 7 days, about 8 days, about 9 days, about 10 days, about 14 days, about 21 days, about 28 days, or about a month.

The term “multi-block” as used herein is meant to refer to the presence of at least two distinct pre-polymer segments in a polymer chain.

Certain terms relating to polymer chemistry, polymer compositions, and methods of polymer microparticle or microsphere formation, as used herein, are defined in WO 2013/015685 A1, WO 2005/068533 A1, and WO 2020/071912 A1.

It is understood that wherever aspects are described herein with the language “comprising,” otherwise analogous aspects described in terms of “consisting of” and/or “consisting essentially of” are also provided.

It is further understood that headers (e.g., “Method of Producing Stable Pharmaceutically Acceptable Formulation”) are provided herein merely for ease of reading, and that wherever aspects are described under a header, the header is not intended to limit the scope of the disclosure. As a non-limiting example, an aspect describing a suitable polymer under a header introducing a pre-filled injector should not be interpreted as limiting the use of that suitable polymer to only pre-filled injectors, unless the aspect itself specifies use of the suitable polymer in a pre-filled injector (e.g., “in some aspects, the pre-filled injector comprises a suitable polymer”). As another non-limiting example, “in some aspects, the antioxidant is sodium metabisulfite” under a header for pre-filled injectors is nevertheless intended to be applicable to methods or compositions unrelated to pre-filled injectors describing the use of one or more antioxidants.

### Apomorphine or a Pharmaceutically Acceptable Salt Thereof

The term “apomorphine” (or “apomorphine free base”) refers to (R)-5,6,6a,7-tetrahydro-6-methyl-4H-dibenzo-[de,g]quinoline-10,11-diol. Apomorphine has the following chemical structure:

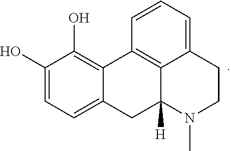 [SMILES: CN1CCc2cccc3c2[C@H]1Cc1ccc(O)c(O)c1-3]

The present disclosure provides a pharmaceutically acceptable formulation of apomorphine free base or a pharmaceutically acceptable salt thereof.

### Microsphere Comprising Apomorphine or a Pharmaceutically Acceptable Salt Thereof

In some aspects, the present disclosure provides a microsphere comprising a first biodegradable polymer and an active drug load of apomorphine or a pharmaceutically acceptable salt thereof.

In some aspects, the microsphere is prepared by a method described herein.

In some aspects, the microsphere comprises one or more polymers described herein.

### Stable Pharmaceutically Acceptable Formulation

In some aspects, the present disclosure provides a stable pharmaceutically acceptable formulation comprising a microsphere, the microsphere comprising a first biodegradable polymer and an active drug load of apomorphine or a pharmaceutically acceptable salt thereof.

In some aspects, the pharmaceutically acceptable salt is an acid addition salt of apomorphine.

In some aspects, the pharmaceutically acceptable salt is apomorphine hydrochloride.

In some aspects, the stable pharmaceutically acceptable formulation further comprises one or more antioxidants. In some aspects, the one or more antioxidants comprises sodium metabisulfite. In some aspects, the antioxidant is sodium metabisulfite. In some aspects, the one or more antioxidants comprises sodium ascorbate. In some aspects, the antioxidant is sodium ascorbate.

In some aspects, the microsphere further comprises one or more additional biodegradable polymers.

In some aspects, the first biodegradable polymer is a copolymer. In some aspects, the first biodegradable polymer is a multi-block copolymer. In some aspects, the multi-block copolymer comprises at least one hydrolysable pre-polymer (A) segment and at least one hydrolysable pre-polymer (B) segment, wherein the segments are linked by a multifunctional chain extender, and wherein the segments are randomly and non-alternatingly distributed over the polymer chain.

In some aspects, the multi-block copolymer has a T_{g} of about 37° C. or less and a Tₘ of about 110° C. to about 250° C. under physiological conditions, and wherein the pre-polymer (A) segment comprises polyethylene glycol.

In some aspects, the polyethylene glycol has a Mₙ of about 150 to about 5000 g/mol. In some aspects, the polyethylene glycol has a Mₙ of about 200 g/mol to about 1500 g/mol. In some aspects, the polyethylene glycol has a Mₙ of about 600 to about 1000 g/mol. In some aspects, the polyethylene glycol has a Mₙ of about 400 to about 3000 g/mol. In some aspects, the polyethylene glycol has a Mₙ of about 600 to about 1500 g/mol. In some aspects, the polyethylene glycol has a Mₙ of about 600 to about 5000 g/mol. In some aspects, the polyethylene glycol has a Mₙ of about 1000 to about 3000 g/mol.

In some aspects, the multi-block copolymer is amorphous and has a glass transition temperature of 37° C. or less at physiological conditions.

In some aspects, the pre-polymer (A) segment and/or the pre-polymer (B) segment comprises one or more linkages selected from the group consisting of: ester linkages, carbonate linkages, anhydride linkages, ether linkages, and combinations thereof. In some aspects, the pre-polymer (A) segment comprises one or more polyether groups. In some aspects, the one or more polyether groups are selected from the group consisting of: polyethylene glycol, polyethylene glycol-polypropylene glycol, polytetramethylene ether glycol, and combinations thereof. In some aspects, said polyether group is polyethylene glycol. In some aspects, a polyether is present as an additional pre-polymer in the multi-block copolymer.

In some aspects, the pre-polymer (A) segment comprises products of a reaction of at least one cyclic monomer with at least one non-cyclic initiator selected from the group consisting of diols, dicarboxylic acids and hydroxycarboxylic acids. In some aspects, the at least one cyclic monomer is selected from the group consisting of glycolide, lactide (D and/or L), ε-caprolactone, δ-valerolactone, trimethylene carbonate, 1,4-dioxane-2-one (para-dioxanone), 1,5-dioxanone-2-one, and a cyclic anhydride. In some aspects, the at least one non-cyclic initiator is selected from the group consisting of succinic acid, glutaric acid, adipic acid, sebacic acid, lactic acid, glycolic acid, ethylene glycol, diethylene glycol, 1,4-butanediol, and 1,6-hexanediol.

In some aspects, the pre-polymer (A) segment comprises reactions products of ester forming monomers selected from diols, dicarboxylic acids, and hydroxycarboxylic acids, preferably the pre-polymer (A) segment comprises reaction products of glycolide, lactide (D and/or L), ε-caprolactone, and/or δ-valerolactone.

In some aspects, the content of pre-polymer (A) in the multi-block copolymer is from about 1% to about 90% based on total weight of the multi-block copolymer. In some aspects, the content of pre-polymer (A) in the multi-block copolymer is from about 2% to about 80%. In some aspects, the content of pre-polymer (A) in the multi-block copolymer is from about 3% to about 70%. In some aspects, the content of pre-polymer (A) in the multi-block copolymer is from about 4% to about 60%. In some aspects, the content of pre-polymer (A) in the multi-block copolymer is from about 5% to about 50%. In some aspects, the content of pre-polymer (A) in the multi-block copolymer is from about 6% to about 40%. In some aspects, the content of pre-polymer (A) in the multi-block copolymer is from about 8% to about 30%. In some aspects, the content of pre-polymer (A) in the multi-block copolymer is from about 10% to about 20%.

In some aspects, the pre-polymer (A) segment has a Mₙ of about 500 g/mol or more. In some aspects, the pre-polymer (A) segment has a Mₙ of about 700 g/mol or more. In some aspects, the pre-polymer (A) segment has a Mₙ of about 1000 g/mol or more. In some aspects, the pre-polymer (A) segment has a Mₙ of about 2000 g/mol or more. In some aspects, the pre-polymer (A) segment has a Mₙ of about 3000 g/mol or more. In some aspects, the pre-polymer (A) segment has a Mₙ of about 4000 g/mol or more.

In some aspects, the pre-polymer (B) segment comprises a polymer derived from hydroxyalkanoate, glycolide lactide (D and/or L), ε-caprolactone, δ-valerolactone, trimethylene carbonate, 1,4-dioxane-2-one or combinations thereof.

In some aspects, the pre-polymer (B) segment comprises poly(glycolide-co-L lactide). In some aspects, the pre-polymer (B) segment comprises poly(glycolide-co-L lactide) with a Mₙ of about 1000 g/mol or more. In some aspects, the pre-polymer (B) segment comprises poly(glycolide-co-L lactide) with a Mₙ of about 2000 g/mol or more. In some aspects, the pre-polymer (B) segment comprises poly(glycolide-co-L lactide) with a Mₙ of about 3000 g/mol or more. In some aspects, the pre-polymer (B) segment comprises poly(glycolide-co-L lactide) with a Mₙ of about 4000 g/mol or more.

In some aspects, the pre-polymer (B) segment comprises a molar amount of about 1% to about 90% of glycolide relative to combined molar amount of glycolide and L-lactide. In some aspects, the pre-polymer (B) segment comprises a molar amount of about 2% to about 50%. In some aspects, the pre-polymer (B) segment comprises a molar amount of about 5% to about 30%. In some aspects, the pre-polymer (B) segment comprises a molar amount of about 10% to about 20%.

In some aspects, the multi-block copolymer comprises from about 10% to about 99% of the pre-polymer (B) segment relative to the total weight of the multi-block copolymer. In some aspects, the multi-block copolymer comprises from about 20% to about 98% of the pre-polymer (B) segment relative to the total weight of the multi-block copolymer. In some aspects, the multi-block copolymer comprises from about 30% to about 97% of the pre-polymer (B) segment relative to the total weight of the multi-block copolymer. In some aspects, the multi-block copolymer comprises from about 40% to about 96% of the pre-polymer (B) segment relative to the total weight of the multi-block copolymer. In some aspects, the multi-block copolymer comprises from about 50% to about 95% of the pre-polymer (B) segment relative to the total weight of the multi-block copolymer. In some aspects, the multi-block copolymer comprises from about 60% to about 94% of the pre-polymer (B) segment relative to the total weight of the multi-block copolymer. In some aspects, the multi-block copolymer comprises from about 70% to about 92% of the pre-polymer (B) segment relative to the total weight of the multi-block copolymer. In some aspects, the multi-block copolymer comprises from about 80% to about 90% of the pre-polymer (B) segment relative to the total weight of the multi-block copolymer. In some aspects, the multi-block copolymer comprises from about 82% to about 88% of the pre-polymer (B) segment relative to the total weight of the multi-block copolymer.

In some aspects, the multifunctional chain extender is a difunctional aliphatic chain extender. In some aspects, the difunctional aliphatic chain extender is a diisocyanate. In some aspects, the diisocyanate is 1,4-butane diisocyanate.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not substantially identical in composition to the second biodegradable polymer.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w. In some aspects, the active drug load of apomorphine or a pharmaceutically acceptable salt thereof in the microspheres is about 30%, about 35%, about 40%, about 45%, about 50%, about 55%, about 60%, about 65%, or about 70% w/w.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microspheres is about 40% w/w or less. In some aspects, the active drug load of apomorphine or a pharmaceutically acceptable salt thereof in the microspheres is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less. In some aspects, the active drug load of apomorphine or a pharmaceutically acceptable salt thereof in the microspheres is about 50% w/w or more. In some aspects, the active drug load of apomorphine or a pharmaceutically acceptable salt thereof in the microspheres is about 40% w/w or more. In some aspects, the active drug load of apomorphine or a pharmaceutically acceptable salt thereof in the microspheres is about 30% w/w or more. In some aspects, the active drug load of apomorphine or a pharmaceutically acceptable salt thereof in the microspheres is about 20% w/w or more. In some aspects, the active drug load of apomorphine or a pharmaceutically acceptable salt thereof in the microspheres is about 10% w/w or more. In some aspects, the active drug load of apomorphine or a pharmaceutically acceptable salt hereof in the microspheres is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 35% w/w, about 40% w/w, about 45% w/w, about 50% w/w, or about 55% w/w.

In some aspects, the stable pharmaceutically acceptable formulation is substantially sterile. In some aspects, the stable pharmaceutically acceptable formulation is sterile.

In some aspects, the stable pharmaceutically acceptable formulation comprises less than 5 wt % of an apomorphine-derived impurity after sealed storage for 24 months at a temperature of 25° C. In some aspects, the stable pharmaceutically acceptable formulation comprises less than 2% wt/wt of an apomorphine-derived impurity after sealed storage for 24 months at a temperature of 25° C. In some aspects, the stable pharmaceutically acceptable formulation comprises less than 1% wt/wt of an apomorphine-derived impurity after sealed storage for 24 months at a temperature of 25° C.

In some aspects, the stable pharmaceutically acceptable formulation is substantially free of an apomorphine-derived impurity after sealed storage for 24 months at a temperature of 25° C.

In some aspects, the apomorphine-derived impurity is an apomorphine oxidation product.

In some aspects, the stable pharmaceutically acceptable formulation has a shelf life of about 14 days at 25° C. following refrigeration. In some aspects, the stable pharmaceutically acceptable formulation has a shelf life of about 7-21 days at 25° C. following refrigeration. In some aspects, the stable pharmaceutically acceptable formulation has a shelf life of about 7 days, about 8 days, about 9 days, about 10 days, about 11 days, about 12 days, about 13 days, about 14 days, about 15 days, about 16 days, about 17 days, about 18 days about 19 days, about 20 days, or about 21 days at 25° C. following refrigeration.

In some aspects, the stable pharmaceutically acceptable formulation has a shelf life of about 24 months at 25° C. In some aspects, the stable pharmaceutically acceptable formulation has a shelf life of about 12-36 months at 25° C. In some aspects, the stable pharmaceutically acceptable formulation has a shelf life of about 12 months, about 13 months, about 14 months, about 15 months, about 16 months, about 17 months, about 18 months, about 19 months, about 20 months, about 21 months, about 22 months, about 23 months, or about 24 months at 25° C.

In some aspects, the stable pharmaceutically acceptable formulation is stored in a sterile container of about 3 mL capacity. In some aspects, the stable pharmaceutically acceptable formulation is stored in a sterile container of about 1-20 mL capacity. In some aspects, the stable pharmaceutically acceptable formulation is stored in a sterile container of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL capacity.

In some aspects, the stable pharmaceutically acceptable formulation is provided in an injector.

In some aspects, the injector is a disposable pen injector.

In some aspects, the stable pharmaceutically acceptable formulation is suitable for parenteral administration. In some aspects, the stable pharmaceutically acceptable formulation is suitable for subcutaneous administration. In some aspects, the stable pharmaceutically acceptable formulation is suitable for intramuscular administration. In some aspects, the stable pharmaceutically acceptable formulation is suitable for intravenous administration.

In some aspects, the amount of apomorphine or a pharmaceutically acceptable salt thereof per dose is about 40 mg to about 200 mg. In some aspects, the amount of apomorphine or a pharmaceutically acceptable salt thereof per dose is about 80 mg to about 100 mg. In some aspects, the amount of apomorphine or a pharmaceutically acceptable salt thereof per dose is about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg.

### Method of Producing Stable Pharmaceutically Acceptable Formulation

In some aspects, the present disclosure provides a method of producing a stable pharmaceutically acceptable formulation comprising a microsphere. In a further aspect, the method comprises providing a first phase comprising: a first biodegradable polymer and an active drug load of apomorphine or a pharmaceutically acceptable salt thereof. In a further aspect, the method comprises adding a second phase comprising an aqueous surfactant continuously into the first phase to form an emulsion. In a further aspect, the method comprises adding a quench solution to the emulsion to produce a volume comprising a microsphere. In a further aspect, the method provides washing, filtering, and drying the microsphere to reduce solvent content.

In some aspects, the present disclosure provides a method of producing a stable pharmaceutically acceptable formulation comprising a microsphere. In a further aspect, the method comprises providing a first phase comprising: a first biodegradable polymer and a solvent system suitable to dissolve the polymer, wherein apomorphine or a pharmaceutically acceptable salt thereof is dispersed but not substantially dissolved in the solvent system. In a further aspect, the method comprises combining the first phase with a second phase comprising an aqueous solution, which comprises a surfactant, thereby forming an emulsion.

In some aspects, the present disclosure provides a method of producing a stable pharmaceutically acceptable formulation comprising a microsphere. In a further aspect, the method comprises providing a first phase comprising: a first biodegradable polymer; an active drug load of apomorphine or a pharmaceutically acceptable salt thereof, and a solvent system suitable to dissolve the polymer and apomorphine. In a further aspect, the method comprises emulsifying the first phase with a second phase, thereby forming an emulsion. In a further aspect, the second phase comprises an aqueous solution which comprises a surfactant. In a further aspect, the method comprises removing a substantial portion of the solvent system from the emulsion, thereby obtaining a microsphere.

In some aspects, the method comprises collecting and drying the microspheres.

In some aspects, emulsification comprises membrane emulsification. In some aspects, the step of emulsifying the first phase with the second phase comprises membrane emulsification of the first phase into the second phase. In some aspects, the step of emulsifying the first phase with the second phase comprises membrane emulsification using a membrane through which the first phase is introduced into the second phase.

In some aspects, the step of removing the substantial portion of the solvent system from the emulsion comprises extraction of the solvent system by the aqueous solution that comprises a surfactant. In some aspects, the step of removing the substantial portion of the solvent system from the emulsion comprises extraction of a first part of the solvent system by the aqueous solution that comprises a surfactant, followed by evaporation of a second part of the solvent system.

In some aspects, the substantial portion of the solvent system is about 80%, about 81%, about 82%, about 83%, about 84%, about 85%, about 86%, about 87%, about 88%, about 89%, about 90%, about 91%, about 92%, about 93%, about 95%, about 95%, about 96%, about 97%, about 98%, about 99%, or about 100% of the solvent system in the emulsion.

In some aspects, the method further comprises washing and/or filtering the microspheres.

In some aspects, the method further comprises drying the microspheres. In some aspects, the drying of the microspheres comprises one or more of lyophilization, vacuum-drying, and freeze-vacuum drying.

In some aspects, the method comprises a step of hardening the microspheres.

In some aspects, the pharmaceutically acceptable salt is an acid addition salt of apomorphine.

In some aspects, the pharmaceutically acceptable salt is apomorphine hydrochloride.

In some aspects, the first phase comprises a solvent system in which apomorphine or a pharmaceutically acceptable salt thereof has a solubility of about 25 mg/mL or more. In some aspects, the first phase comprises a solvent system in which apomorphine or a pharmaceutically acceptable salt thereof has a solubility of about 50 mg/mL or more. In some aspects, the first phase comprises a solvent system in which apomorphine or a pharmaceutically acceptable salt thereof has a solubility of about 100 mg/mL or more. In some aspects, the first phase comprises a solvent system in which apomorphine or a pharmaceutically acceptable salt thereof has a solubility of about 200 mg/mL or more.

In some aspects, the first phase comprises one or more solvents selected from the group consisting of: dichloromethane, ethyl acetate, chloroform, methanol, benzyl alcohol, dimethyl formamide, dimethyl sulfoxide, N-methyl pyrrolidone, and dimethyl acetamide. In some aspects, the first phase comprises dichloromethane. In some aspects, the first phase comprises ethyl acetate. In some aspects, the first phase comprises chloroform. In some aspects, the first phase comprises methanol. In some aspects, the first phase comprises benzyl alcohol. In some aspects, the first phase comprises dimethyl formamide. In some aspects, the first phase comprises dimethyl sulfoxide. In some aspects, the first phase comprises N-methyl pyrrolidone. In some aspects, the first phase comprises dimethyl acetamide.

In some aspects, the first phase comprises a first solvent and a second solvent. In some aspects, the first solvent is present in an amount of about 1% to about 99% relative to the combined volume of the first solvent and the second solvent. In some aspects, the first solvent is present in an amount of about 5% to about 50% relative to the combined volume of the first solvent and the second solvent. In some aspects, the first solvent is present in an amount of about 10% to about 40% relative to the combined volume of the first solvent and the second solvent. In some aspects, the first solvent is present in an amount of about 15% to about 30% relative to the combined volume of the first solvent and the second solvent. In some aspects, the first solvent is present in an amount of about 20% to about 25% relative to the combined volume of the first solvent and the second solvent. In some aspects, the first solvent is present in an amount of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% relative to the combined volume of the first solvent and the second solvent.

In some aspects, the first phase comprises dichloromethane in combination with one or more additional solvents. In some aspects, the one or more additional solvents are selected from the group consisting of: dimethyl formamide, dimethyl sulfoxide, and N-methyl pyrrolidone. In some aspects, the first phase comprises dichloromethane and dimethyl sulfoxide. In some aspects, dimethyl sulfoxide is present in an amount of about 5% to about 50% relative to the combined volume of dimethyl sulfoxide and dichloromethane in the first phase. In some aspects, dimethyl sulfoxide is present in an amount of about 10% to about 40% relative to the combined volume of dimethyl sulfoxide and dichloromethane in the first phase. In some aspects, dimethyl sulfoxide is present in an amount of about 15% to about 30% relative to the combined volume of dimethyl sulfoxide and dichloromethane in the first phase. In some aspects, dimethyl sulfoxide is present in an amount of about 20% to about 25% relative to the combined volume of dimethyl sulfoxide and dichloromethane in the first phase.

In some aspects, the first phase comprises about 2% to about 25% by weight of the combined mass of the first biodegradable polymer and the one or more additional biodegradable polymers.

In some aspects, the first phase comprises about 2% to about 25% by weight of apomorphine or a pharmaceutically acceptable salt thereof.

In some aspects, the first phase comprises one or more antioxidants. In some aspects, the one or more antioxidants comprises sodium metabisulfite or sodium ascorbate. In some aspects, the one or more antioxidants comprises sodium metabisulfite or sodium ascorbate in an amount from about 0.01% to about 5% w/v relative to the first phase. In some aspects, the one or more antioxidants comprises sodium metabisulfite or sodium ascorbate in an amount from about 0.05% to about 2% w/v relative to the first phase. In some aspects, the one or more antioxidants comprises sodium metabisulfite or sodium ascorbate in an amount from about 0.1% to about 1% w/v relative to the first phase.

In some aspects, the second phase comprises polyvinyl alcohol.

In some aspects, the second phase comprises sodium chloride.

In some aspects, the second phase comprises one or more antioxidants.

In some aspects, the one or more antioxidants comprises sodium metabisulfite. In some aspects, the one or more antioxidants comprises sodium metabisulfite in an amount of about 0.1% to about 1% w/v in the second phase. In some aspects, the one or more antioxidants comprises sodium metabisulfite in an amount of about 0.15% w/v in the second phase. In some aspects, the one or more antioxidants comprises sodium metabisulfite in an amount of about 0.1%, about 0.2%, about 0.3%, about 0.4%, about 0.5%, about 0.6%, about 0.7%, about 0.8%, about 0.9%, or about 1.0% w/v in the second phase.

In some aspects, the one or more antioxidants comprises sodium ascorbate. In some aspects, the one or more antioxidants comprises sodium ascorbate in an amount of about 0.1% to about 1% w/v in the second phase. In some aspects, the one or more antioxidants comprises sodium ascorbate in an amount of about 0.15% w/v in the second phase. In some aspects, the one or more antioxidants comprises sodium ascorbate in an amount of about 0.1%, about 0.2%, about 0.3%, about 0.4%, about 0.5%, about 0.6%, about 0.7%, about 0.8%, about 0.9%, or about 1.0% w/v in the second phase.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the aqueous surfactant comprises one or more of a cationic surfactant, an anionic surfactant, or a non-ionic surfactant.

In some aspects, the second phase further comprises one or more of: a buffer solution, one or more agents for adjusting the viscosity of the aqueous surfactant, and an agent for adjusting the ionic strength of the solution.

In some aspects, the first phase is stirred prior to and/or during the addition of the second phase.

In some aspects, the emulsion is stirred prior to and/or during the addition of the quench solution.

In some aspects, the volume comprising a microsphere is stirred prior to and/or during any of the steps of washing, filtering and drying the microsphere.

In some aspects, the first biodegradable polymer is substantially enclosed by a second biodegradable polymer.

In some aspects, the first biodegradable polymer is not identical in composition to the second biodegradable polymer.

In some aspects, the first phase is prepared by mixing a solution comprising the first biodegradable polymer with a solution comprising the apomorphine or a pharmaceutically acceptable salt thereof.

In some aspects, the first biodegradable polymer and/or the second biodegradable polymer is dissolved in a solvent highly or fully miscible with water selected from the group consisting of: dimethyl sulfoxide, N-methyl-2-pyrrolidone, tetrahydrofuran, tetraglycol, acetone, an acetone/methyl ethyl ketone mixture, an acetone/methyl acetate mixture, a tetrahydrofuran/ethyl acetate mixture, and a tetrahydrofuran/ethyl formate mixture.

In some aspects, the solvent highly or fully miscible with water is an acetone/methyl ethyl ketone mixture.

In some aspects, the acetone/methyl ethyl ketone mixture comprises about 70% acetone and about 30% methyl ethyl ketone, by volume.

In some aspects, the first biodegradable polymer and/or the second biodegradable polymer is dissolved in a solvent having limited water solubility selected from the group consisting of: ethyl acetate, methyl acetate, ethyl formate, propyl formate, isopropyl formate, methyl ethyl ketone, and a mixture of two or more thereof.

### Pre-Filled Injector

In some aspects, the present disclosure provides a pre-filled injector, the pre-filled injector comprising a stable pharmaceutically acceptable formulation. In a further aspect, the formulation comprises a microsphere. In a further aspect, the microsphere comprises a first biodegradable polymer and an active drug load of apomorphine or a pharmaceutically acceptable salt thereof.

In some aspects, the pharmaceutically acceptable salt is an acid addition salt of apomorphine.

In some aspects, the pharmaceutically acceptable salt is apomorphine hydrochloride.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not identical in composition to the second biodegradable polymer.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w. In some aspects, the active drug load of apomorphine or a pharmaceutically acceptable salt thereof in the microspheres is about 30%, about 35%, about 40%, about 45%, about 50%, about 55%, about 60%, about 65%, or about 70% w/w.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microspheres is about 40% w/w or less. In some aspects, the active drug load of apomorphine or a pharmaceutically acceptable salt thereof in the microspheres is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less. In some aspects, the active drug load of apomorphine or a pharmaceutically acceptable salt thereof in the microspheres is about 50% w/w or more. In some aspects, the active drug load of apomorphine or a pharmaceutically acceptable salt thereof in the microspheres is about 40% w/w or more. In some aspects, the active drug load of apomorphine or a pharmaceutically acceptable salt thereof in the microspheres is about 30% w/w or more. In some aspects, the active drug load of apomorphine or a pharmaceutically acceptable salt thereof in the microspheres is about 20% w/w or more. In some aspects, the active drug load of apomorphine or a pharmaceutically acceptable salt thereof in the microspheres is about 10% w/w or more. In some aspects, the active drug load of apomorphine or a pharmaceutically acceptable salt thereof in the microspheres is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 35% w/w, about 40% w/w, about 45% w/w, about 50% w/w, or about 55% w/w.

In some aspects, the stable pharmaceutical formulation is substantially sterile. In some aspects, the stable pharmaceutically acceptable formulation is sterile.

In some aspects, the stable pharmaceutical formulation has a shelf life of about 14 days at 25° C. following refrigeration. In some aspects, the stable pharmaceutical formulation has a shelf life of about 7-21 days at 25° C. following refrigeration. In some aspects, the stable pharmaceutical formulation has a shelf life of about 7 days, about 8 days, about 9 days, about 10 days, about 11 days, about 12 days, about 13 days, about 14 days about 15 days, about 16 days, about 17 days, about 18 days about 19 days, about 20 days, or about 21 days at 25° C. following refrigeration.

In some aspects, the stable pharmaceutical formulation has a shelf life of about 24 months at 25° C. In some aspects, the stable pharmaceutical formulation has a shelf life of about 12-36 months at 25° C. In some aspects, the stable pharmaceutical formulation has a shelf life of about 12 months, about 13 months, about 14 months, about 15 months, about 16 months, about 17 months, about 18 months, about 19 months, about 20 months, about 21 months, about 22 months, about 23 months, or about 24 months at 25° C.

In some aspects, the pre-filled injector is a pen injector or an autoinjector.

In some aspects, the pre-filled injector is disposable.

In some aspects, the pre-filled injector is a disposable pen injector.

In some aspects, the pre-filled injector comprises a cartridge comprising the stable pharmaceutically acceptable formulation.

In some aspects, the cartridge is a dual chamber cartridge.

In some aspects, the dual chamber cartridge comprises, in a first chamber, a first volume comprising a microsphere, the microsphere comprising an active drug load of apomorphine or a pharmaceutically acceptable salt thereof. In some aspects, the dual chamber cartridge comprises, in a second chamber, a second volume comprising a dilution medium.

In some aspects, the first volume and the second volume can be combined to produce a pharmaceutically acceptable formulation disclosed herein.

In some aspects, the pre-filled injector is configurable to combine the first volume and the second volume prior to injection. In some aspects, the pre-filled injector is configurable to combine the first volume and the second volume during injection.

In some aspects, the pre-filled injector is configurable to dispense the first volume and the second volume in about equal amounts by volume. In some aspects, the pre-filled injector is configurable to dispense the first volume and the second volume in amounts that are not equal by volume.

In some aspects, the first volume has a shelf life of about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 days at 25° C. following refrigeration. In some aspects, the first volume has a shelf life of about 7 days at 25° C. following refrigeration. In some aspects, the first volume has a shelf life of about 14 days at 25° C. following refrigeration. In some aspects, the first volume has a shelf life of about 21 days at 25° C. following refrigeration.

In some aspects, the first volume has a shelf life of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 months at 25° C. In some aspects, the first volume has a shelf life of about 3 months at 25° C. In some aspects, the first volume has a shelf life of about 6 months at 25° C. In some aspects, the first volume has a shelf life of about 12 months at 25° C.

In some aspects, the second volume has a shelf life of about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 days at 25° C. following refrigeration. In some aspects, the second volume has a shelf life of about 7 days at 25° C. following refrigeration. In some aspects, the second volume has a shelf life of about 14 days at 25° C. following refrigeration. In some aspects, the second volume has a shelf life of about 21 days at 25° C. following refrigeration.

In some aspects, the second volume has a shelf life of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 months at 25° C. In some aspects, the second volume has a shelf life of about 3 months at 25° C. In some aspects, the second volume has a shelf life of about 6 months at 25° C. In some aspects, the second volume has a shelf life of about 12 months at 25° C.

In some aspects, the pre-filled injector comprises a 18 G to 30 G needle. In some aspects, the pre-filled injector comprises a 21 G needle. In some aspects, the pre-filled injector comprises a 23 G needle. In some aspects, the pre-filled injector comprises a 25 G needle.

In some aspects, the pre-filled injector does not comprise a needle.

In some aspects, the amount of the stable pharmaceutically acceptable formulation dispensed in a single injection contains about 40 mg to about 200 mg of apomorphine or a pharmaceutically acceptable salt thereof.

In some aspects, the amount of the stable pharmaceutically acceptable formulation dispensed in a single injection contains about 40 mg to about 200 mg of apomorphine or a pharmaceutically acceptable salt thereof. In some aspects, the amount of the stable pharmaceutically acceptable formulation dispensed in a single injection contains about 80 mg to about 100 mg of apomorphine or a pharmaceutically acceptable salt thereof. In some aspects, the amount of the stable pharmaceutically acceptable formulation dispensed in a single injection contains about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of apomorphine or a pharmaceutically acceptable salt thereof. In some aspects, the amount of the stable pharmaceutical formulation dispensed in a single injection contains about 90 mg of apomorphine or a pharmaceutically acceptable salt thereof.

### Method of Manufacturing Pre-Filled Injector

In some aspects, the present disclosure provides a method of manufacturing a pre-filled injector comprising a stable pharmaceutically acceptable formulation. In a further aspect, the method comprises preparing a stable pharmaceutically acceptable formulation. In a further aspect, the formulation comprises a microsphere. In a further aspect, the microsphere comprises a first biodegradable polymer and an active drug load of apomorphine or a pharmaceutically acceptable salt thereof. In a further aspect, the method comprises loading a sterile cartridge with the stable pharmaceutically acceptable formulation. In a further aspect, the method comprises attaching the sterile cartridge operably to an injector.

In some aspects, the pharmaceutically acceptable salt is an acid addition salt of apomorphine.

In some aspects, the pharmaceutically acceptable salt is apomorphine hydrochloride.

In some aspects, the first biodegradable polymer is selected from the group consisting of: a polylactide, a polyglycolide, and a poly(lactide-co-glycolide) copolymer. In some aspects, the first biodegradable polymer comprises one or more of: a polylactide, a polyglycolide, a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not identical in composition to the second biodegradable polymer.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w. In some aspects, the active drug load of apomorphine or a pharmaceutically acceptable salt thereof in the microspheres is about 30%, about 35%, about 40%, about 45%, about 50%, about 55%, about 60%, about 65%, or about 70% w/w.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microspheres is about 40% w/w or less. In some aspects, the active drug load of apomorphine or a pharmaceutically acceptable salt thereof in the microspheres is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less. In some aspects, the active drug load of apomorphine or a pharmaceutically acceptable salt thereof in the microspheres is about 50% w/w or more. In some aspects, the active drug load of apomorphine or a pharmaceutically acceptable salt thereof in the microspheres is about 40% w/w or more. In some aspects, the active drug load of apomorphine or a pharmaceutically acceptable salt thereof in the microspheres is about 30% w/w or more. In some aspects, the active drug load of apomorphine or a pharmaceutically acceptable salt thereof in the microspheres is about 20% w/w or more. In some aspects, the active drug load of apomorphine or a pharmaceutically acceptable salt thereof in the microspheres is about 10% w/w or more. In some aspects, the active drug load of apomorphine or a pharmaceutically acceptable salt thereof in the microspheres is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, or about 30% w/w, about 35% w/w, about 40% w/w, about 45% w/w, about 50% w/w, or about 55% w/w.

In some aspects, the stable pharmaceutical formulation is substantially sterile. In some aspects, the stable pharmaceutical formulation is sterile.

In some aspects, the stable pharmaceutical formulation has a shelf life of about 14 days at 25° C. following refrigeration. In some aspects, the stable pharmaceutical formulation has a shelf life of about 7-21 days at 25° C. following refrigeration. In some aspects, the stable pharmaceutical formulation has a shelf life of about 7 days, about 8 days, about 9 days, about 10 days, about 11 days, about 12 days, about 13 days, about 14 days about 15 days, about 16 days, about 17 days, about 18 days about 19 days, about 20 days, or about 21 days at 25° C. following refrigeration.

In some aspects, the stable pharmaceutical formulation has a shelf life of about 24 months at 25° C. In some aspects, the stable pharmaceutical formulation has a shelf life of about 12-36 months at 25° C. In some aspects, the stable pharmaceutical formulation has a shelf life of about 12 months, about 13 months, about 14 months, about 15 months, about 16 months, about 17 months, about 18 months, about 19 months, about 20 months, about 21 months, about 22 months, about 23 months, or about 24 months at 25° C.

In some aspects, the pre-filled injector is a pen injector or an autoinjector.

In some aspects, the pre-filled injector is disposable.

In some aspects, the pre-filled injector is a disposable pen injector.

In some aspects, the pre-filled injector comprises a cartridge comprising the stable pharmaceutically acceptable formulation.

In some aspects, the cartridge is a dual chamber cartridge.

In some aspects, the dual chamber cartridge comprises, in a first chamber, a first volume comprising a microsphere, the microsphere comprising an active drug load of apomorphine or a pharmaceutically acceptable salt thereof. In some aspects, the dual chamber cartridge comprises, in a second chamber, a second volume comprising a dilution medium.

In some aspects, the first volume and the second volume can be combined to produce a pharmaceutically acceptable formulation disclosed herein.

In some aspects, the pre-filled injector is configurable to combine the first volume and the second volume prior to injection. In some aspects, the pre-filled injector is configurable to combine the first volume and the second volume during injection.

In some aspects, the pre-filled injector is configurable to dispense the first volume and the second volume in about equal amounts by volume. In some aspects, the pre-filled injector is configurable to dispense the first volume and the second volume in amounts that are not equal by volume.

In some aspects, the pre-filled injector comprises a 18 G to 30 G needle. In some aspects, the pre-filled injector comprises a 21 G needle. In some aspects, the pre-filled injector comprises a 23 G needle. In some aspects, the pre-filled injector comprises a 25 G needle.

In some aspects, the pre-filled injector does not comprise a needle.

In some aspects, the amount of the stable pharmaceutically acceptable formulation dispensed in a single injection contains about 40 mg to about 200 mg of apomorphine or a pharmaceutically acceptable salt thereof. In some aspects, the amount of the stable pharmaceutically acceptable formulation dispensed in a single injection contains about 80 mg to about 100 mg of apomorphine or a pharmaceutically acceptable salt thereof. In some aspects, the amount of the stable pharmaceutically acceptable formulation dispensed in a single injection contains about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of apomorphine or a pharmaceutically acceptable salt thereof. In some aspects, the amount of the stable pharmaceutical formulation dispensed in a single injection contains about 90 mg.

### Method of Treatment

In some aspects, the present disclosure provides a method of treating motor symptoms associated with Parkinson's disease, the method comprising administering to a subject in need thereof a stable pharmaceutically acceptable formulation. In a further aspect, the formulation comprises microspheres. In a further aspect, the microspheres comprise a first biodegradable polymer and an active drug load of apomorphine or a pharmaceutically acceptable salt thereof.

In some aspects, the pharmaceutically acceptable salt is an acid addition salt of apomorphine.

In some aspects, the pharmaceutically acceptable salt is apomorphine hydrochloride.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w. In some aspects, the active drug load of apomorphine or a pharmaceutically acceptable salt thereof in the microspheres is about 30%, about 35%, about 40%, about 45%, about 50%, about 55%, about 60%, about 65%, or about 70% w/w.

In some aspects, the amount of the stable pharmaceutically acceptable formulation administered to the subject contains an amount of apomorphine or a pharmaceutically acceptable salt thereof of about 40 mg to about 200 mg. In some aspects, the amount of the stable pharmaceutically acceptable formulation administered to the subject contains an amount of apomorphine or a pharmaceutically acceptable salt thereof of about 80 mg to about 100 mg. In some aspects, the amount of the stable pharmaceutically acceptable formulation administered to the subject contains an amount of apomorphine or a pharmaceutically acceptable salt thereof of about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In some aspects, the amount of the stable pharmaceutically acceptable formulation administered to the subject contains an amount of apomorphine or a pharmaceutically acceptable salt thereof of about 90 mg.

In some aspects, the stable pharmaceutically acceptable formulation is administered with an injector.

In some aspects, the stable pharmaceutically acceptable formulation is administered parenterally.

In some aspects, the stable pharmaceutically acceptable formulation is administered subcutaneously or intramuscularly.

In some aspects, administration of the stable pharmaceutically acceptable formulation effects a therapeutically effective concentration of apomorphine for about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days following an initial burst of apomorphine in the plasma. In some aspects, administration of the stable pharmaceutically acceptable formulation effects a therapeutically effective concentration of apomorphine for about 7 days following an initial burst of apomorphine in the plasma.

In some aspects, the therapeutically effective concentration of apomorphine is about 10 μg/L, about 20 μg/L, about 30 μg/L, about 40 μg/L, about 50 μg/L, about 60 μg/L, about 70 μg/L, about 80 μg/L, about 90 μg/L, about 100 μg/L, about 110 μg/L, about 120 μg/L, about 130 μg/L, about 140 μg/L, about 150 μg/L, about 160 μg/L, about 170 μg/L, about 180 μg/L, about 190 μg/L, or about 200 μg/L.

In some aspects, administration of the stable pharmaceutically acceptable formulation effects a steady-state plasma concentration of apomorphine following an initial burst of apomorphine in the plasma.

In some aspects, the steady-state plasma concentration is about 35 μg/L. In some aspects, the steady-state plasma concentration is about 20-50 μg/L. In some aspects, the steady-state plasma concentration is about 30-40 μg/L. In some aspects, the steady-state plasma concentration is about 20 μg/L, about 21 μg/L, about 22 μg/L, about 23 μg/L, about 24 μg/L, about 25 μg/L, about 26 μg/L, about 27 μg/L, about 28 μg/L, about 29 μg/L, about 30 μg/L, about 31 μg/L, about 32 μg/L, about 33 μg/L, about 34 μg/L, about 35 μg/L, about 36 μg/L, about 37 μg/L, about 38 μg/L, about 39 μg/L, about 40 μg/L, about 41 μg/L, about 42 μg/L, about 43 μg/L, about 44 μg/L, about 45 μg/L, about 46 μg/L, about 47 μg/L, about 48 μg/L, about 49 μg/L, or about 50 μg/L.

In some aspects, the steady-state plasma concentration is maintained for about 7 days. In some aspects, the steady-state plasma concentration is maintained for about 3-10 days. In some aspects, the steady-state plasma concentration is maintained for about 3 days, about 4 days, about 5 days, about 6 days, about 7 days, about 8 days, about 9 days, or about 10 days.

In some aspects, the initial burst of apomorphine is about 5% of the total dose of apomorphine or a pharmaceutically acceptable salt thereof. In some aspects, the initial burst of apomorphine is about 1-80% of the total dose of apomorphine or a pharmaceutically acceptable salt thereof. In some aspects, the burst of apomorphine is about 20% to about 80% of the total dose of apomorphine or a pharmaceutically acceptable salt thereof in 24 hours. In some aspects, the burst of apomorphine is about 40% of the total dose of apomorphine or a pharmaceutically acceptable salt thereof in 24 hours.

In some aspects, the initial burst of apomorphine is about 1%, about 5%, about 10%, about 15%, about 20%, about 25%, about 30%, about 35%, about 40%, about 45%, about 50%, about 55%, about 60%, about 65%, about 70%, or about 80% of the total dose of apomorphine or a pharmaceutically acceptable salt thereof. In some aspects, the initial burst of apomorphine is about 20-80% of the total dose of apomorphine or a pharmaceutically acceptable salt thereof. In some aspects, the initial burst of apomorphine is about 40-60% of the total dose of apomorphine or a pharmaceutically acceptable salt thereof. In some aspects, the initial burst of apomorphine is about 30-50% of the total dose of apomorphine or a pharmaceutically acceptable salt thereof. In some aspects, the initial burst of apomorphine is about 10-30% of the total dose of apomorphine or a pharmaceutically acceptable salt thereof. In some aspects, the initial burst of apomorphine is about 1-15% of the total dose of apomorphine or a pharmaceutically acceptable salt thereof. In some aspects, the initial burst of apomorphine is about 1%, about 2%, about 3%, about 4%, about 5%, about 6%, about 7%, about 8%, about 9%, about 10%, about 11%, about 12%, about 13%, about 14%, or about 15% of the total dose of apomorphine or a pharmaceutically acceptable salt thereof.

In some aspects, the initial burst of apomorphine is completed about 0.5 to about 24 hours after injection. In some aspects, the initial burst of apomorphine is completed about 1 to about 12 hours after injection. In some aspects, the initial burst of apomorphine is completed about 0.5 hours, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after injection.

In some aspects, the stable pharmaceutically acceptable formulation is administered twice per week. In some aspects, the stable pharmaceutically acceptable formulation is administered once per week. In some aspects, the stable pharmaceutically acceptable formulation is administered once every two weeks. In some aspects, the stable pharmaceutically acceptable formulation is administered once every three weeks. In some aspects, the stable pharmaceutically acceptable formulation is administered once every 28 days. In some aspects, the stable pharmaceutically acceptable formulation is administered once per month.

In some aspects, administration of the stable pharmaceutically acceptable formulation results in less hypotension relative to administration with immediate release subcutaneous apomorphine hydrochloride injection.

In some aspects, administration of the stable pharmaceutically acceptable formulation results in less nausea relative to administration with immediate release subcutaneous apomorphine hydrochloride injection.

In some aspects, administration of the stable pharmaceutically acceptable formulation results in less adverse injection site reactions relative to administration with immediate release subcutaneous apomorphine hydrochloride injection.

In some aspects, the subject in need suffers from Parkinson's disease.

In some aspects, administration of the stable pharmaceutically acceptable formulation is effective in reducing the occurrence, duration, or severity of intermittent episodes of muscle stiffness and/or loss of muscle control associated with Parkinson's disease.

In some aspects, administration of the stable pharmaceutically acceptable formulation is effective at minimizing hypotension or nausea, or reducing hypotension or nausea by at least 50% when compared to parenteral administration of immediate release apomorphine hydrochloride.

In some aspects, injection site reactions are substantially reduced relative to injection site reactions characteristic of injection with immediate release apomorphine hydrochloride.

### Long-Acting Dosage Form

In some aspects, the present disclosure provides a long-acting dosage form comprising microspheres, the microspheres comprising a first biodegradable polymer and about 40 mg to about 200 mg of an active drug load of apomorphine or a pharmaceutically acceptable salt thereof. In a further aspect, administration of a single dose of the long-acting dosage form to a subject results in at least one of the pharmacokinetic parameters selected from the group consisting of: (a) a steady state plasma profile of apomorphine from day 1 to day 7 following administration exhibiting a mean Cₘₐₓ value no greater than the steady state plasma level of apomorphine provided by 2 mg of immediate release subcutaneous injection of apomorphine hydrochloride; (b) an apomorphine elimination half-life of about 40 minutes to about 60 minutes; and (c) a zero-order or a first-order release profile of apomorphine or a pharmaceutically acceptable salt thereof.

In some aspects, administration of a single dose of the long-acting dosage form to a subject results in a zero-order release profile. In some aspects, administration of a single dose of the long-acting dosage form to a subject results in a first-order release profile.

In some aspects, the release profile corresponds to about 1% to about 50% release of the total administered dose of apomorphine or a pharmaceutically acceptable salt thereof per day. In some aspects, the release profile corresponds to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% release of the total administered dose of apomorphine or a pharmaceutically acceptable salt thereof per day. In some aspects, the release profile corresponds to about 3% to about 15% release of the total administered dose of apomorphine or a pharmaceutically acceptable salt thereof per day. In some aspects, the release profile corresponds to about 12% to about 14% release of the total administered dose of apomorphine or a pharmaceutically acceptable salt thereof per day.

In some aspects, the pharmaceutically acceptable salt is an acid addition salt of apomorphine.

In some aspects, the pharmaceutically acceptable salt is apomorphine hydrochloride.

In some aspects, the long-acting dosage form comprises a plurality of microspheres comprising apomorphine or a pharmaceutically acceptable salt thereof, wherein the plurality of microspheres is characterized by a median particle size (“D50”) of about 20 μm to about 80 μm. In some aspects, the plurality of microspheres is characterized by a D50 of about 20 μm, about 25 μm, 30 μm, about 35 μm, about 40 μm, about 45 μm, about 50 μm, about 55 μm, about 60 μm, about 65 μm, about 70 μm, about 75 μm, or about 80 μm. In some aspects, the plurality of microspheres is characterized by a D50 of about 50 μm. In some aspects, the plurality of microspheres is characterized by a D50 of about 70 μm.

In some aspects, the plurality of microspheres is characterized by a D50 having a coefficient of variance (“CV”) less than 70%. In some aspects, the plurality of microspheres is characterized by a D50 having a CV less than 60%. In some aspects, the plurality of microspheres is characterized by a D50 having a CV less than 50%. In some aspects, the plurality of microspheres is characterized by a D50 having a CV less than 40%. In some aspects, the plurality of microspheres is characterized by a D50 having a CV less than 30%. In some aspects, the plurality of microspheres is characterized by a D50 having a CV less than 20%. In some aspects, the plurality of microspheres is characterized by a D50 having a CV less than 10%.

In some aspects, the active drug load of apomorphine or a pharmaceutically acceptable salt thereof in the microspheres is about 20% to about 80% w/w. In some aspects, the active drug load of apomorphine or a pharmaceutically acceptable salt thereof in the microspheres is about 30% to about 70% w/w. In some aspects, the active drug load of apomorphine or a pharmaceutically acceptable salt thereof in the microspheres is about 50% to about 60% w/w. In some aspects, the active drug load of apomorphine or a pharmaceutically acceptable salt thereof in the microspheres is about 40% to about 50% w/w. In some aspects, the active drug load of apomorphine or a pharmaceutically acceptable salt thereof in the microspheres is about 30% to about 40% w/w. In some aspects, the active drug load of apomorphine or a pharmaceutically acceptable salt thereof in the microspheres is about 30%, about 35%, about 40%, about 45%, about 50%, about 55%, about 60%, about 65%, or about 70% w/w.

In some aspects, the amount of the stable pharmaceutically acceptable formulation administered to the subject contains an amount of apomorphine or a pharmaceutically acceptable salt thereof of about 80 mg to about 100 mg. In some aspects, the amount of the stable pharmaceutically acceptable formulation administered to the subject contains an amount of apomorphine or a pharmaceutically acceptable salt thereof of about 90 mg. In some aspects, the amount of the stable pharmaceutically acceptable formulation administered to the subject contains an amount of apomorphine or a pharmaceutically acceptable salt thereof of about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg.

In some aspects, the first biodegradable polymer is selected from the group consisting of: a polylactide, a polyglycolide, and a poly(lactide-co-glycolide) copolymer.

In some aspects, the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

In some aspects, the first biodegradable polymer is substantially enclosed by a second biodegradable polymer.

In some aspects, the first biodegradable polymer is not identical in composition to the second biodegradable polymer.

In some aspects, the active drug load of apomorphine or a pharmaceutically acceptable salt thereof in the microspheres is about 50% w/w or less. In some aspects, the active drug load of apomorphine or a pharmaceutically acceptable salt thereof in the microspheres is about 40% w/w or less. In some aspects, the active drug load of apomorphine or a pharmaceutically acceptable salt thereof in the microspheres is about 30% w/w or less. In some aspects, the active drug load of apomorphine or a pharmaceutically acceptable salt thereof in the microspheres is about 20% w/w or less. In some aspects, the active drug load of apomorphine or a pharmaceutically acceptable salt thereof in the microspheres is about 10% w/w or less. In some aspects, the active drug load of apomorphine or a pharmaceutically acceptable salt thereof in the microspheres is about 50% w/w or more. In some aspects, the active drug load of apomorphine or a pharmaceutically acceptable salt thereof in the microspheres is about 40% w/w or more. In some aspects, the active drug load of apomorphine or a pharmaceutically acceptable salt thereof in the microspheres is about 30% w/w or more. In some aspects, the active drug load of apomorphine or a pharmaceutically acceptable salt thereof in the microspheres is about 20% w/w or more. In some aspects, the active drug load of apomorphine or a pharmaceutically acceptable salt thereof in the microspheres is about 10% w/w or more. In some aspects, the active drug load of apomorphine or a pharmaceutically acceptable salt thereof in the microspheres is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 35% w/w, about 40% w/w, about 45% w/w, about 50% w/w, or about 55% w/w.

In some aspects, the stable pharmaceutical formulation is substantially sterile. In some aspects, the stable pharmaceutical formulation is sterile.

In some aspects, the stable pharmaceutical formulation has a shelf life of about 14 days at 25° C. following refrigeration. In some aspects, the stable pharmaceutical formulation has a shelf life of about 7-21 days at 25° C. following refrigeration. In some aspects, the stable pharmaceutical formulation has a shelf life of about 7 days, about 8 days, about 9 days, about 10 days, about 11 days, about 12 days, about 13 days, about 14 days about 15 days, about 16 days, about 17 days, about 18 days about 19 days, about 20 days, or about 21 days at 25° C. following refrigeration.

In some aspects, the stable pharmaceutical formulation has a shelf life of about 24 months at 25° C. In some aspects, the stable pharmaceutical formulation has a shelf life of about 12-36 months at 25° C. In some aspects, the stable pharmaceutical formulation has a shelf life of about 12 months, about 13 months, about 14 months, about 15 months, about 16 months, about 17 months, about 18 months, about 19 months, about 20 months, about 21 months, about 22 months, about 23 months, or about 24 months at 25° C.

In some aspects, the stable pharmaceutically acceptable formulation is administered from an injector.

In some aspects, the injector is a pen injector or an autoinjector.

In some aspects, wherein the injector is disposable.

In some aspects, the injector is a disposable pen injector.

In some aspects, the injector comprises a cartridge comprising the stable pharmaceutically acceptable formulation.

In some aspects, the cartridge is a dual chamber cartridge.

In some aspects, the dual chamber cartridge comprises, in a first chamber, a first volume comprising a microsphere, the microsphere comprising an active drug load of apomorphine or a pharmaceutically acceptable salt thereof. In some aspects, the dual chamber cartridge comprises, in a second chamber, a second volume comprising a dilution medium.

In some aspects, the first volume and the second volume can be combined to produce a pharmaceutically acceptable formulation disclosed herein.

In some aspects, the pre-filled injector is configurable to combine the first volume and the second volume prior to injection. In some aspects, the pre-filled injector is configurable to combine the first volume and the second volume during injection.

In some aspects, the pre-filled injector is configurable to dispense the first volume and the second volume in about equal amounts by volume. In some aspects, the pre-filled injector is configurable to dispense the first volume and the second volume in amounts that are not equal by volume.

In some aspects, the pre-filled injector comprises a 18 G to 30 G needle. In some aspects, the injector comprises a 21 G needle. In some aspects, the pre-filled injector comprises a 23 G needle. In some aspects, the pre-filled injector comprises a 25 G needle.

In some aspects, the injector is a pre-filled injector comprising the stable pharmaceutically acceptable formulation.

In some aspects, the stable pharmaceutically acceptable formulation is administered parenterally.

In some aspects, the stable pharmaceutically acceptable formulation is administered subcutaneously or intramuscularly.

In some aspects, administration of the stable pharmaceutically acceptable formulation effects a therapeutically effective concentration of apomorphine for about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days following an initial burst of apomorphine in the plasma. In some aspects, administration of the stable pharmaceutically acceptable formulation effects a therapeutically effective concentration of apomorphine for about 7 days following an initial burst of apomorphine in the plasma.

In some aspects, the therapeutically effective concentration of apomorphine is about 10 μg/L, about 20 μg/L, about 30 μg/L, about 40 μg/L, about 50 μg/L, about 60 μg/L, about 70 μg/L, about 80 μg/L, about 90 μg/L, about 100 μg/L, about 110 μg/L, about 120 μg/L, about 130 μg/L, about 140 μg/L, about 150 μg/L, about 160 μg/L, about 170 μg/L, about 180 μg/L, about 190 μg/L, or about 200 μg/L.

In some aspects, administration of the stable pharmaceutically acceptable formulation effects a steady-state plasma concentration of apomorphine following an initial burst of apomorphine in the plasma.

In some aspects, the steady-state plasma concentration is about 35 μg/L. In some aspects, the steady-state plasma concentration is about 20-50 μg/L. In some aspects, the steady-state plasma concentration is about 30-40 μg/L. In some aspects, the steady-state plasma concentration is about 20 μg/L, about 21 μg/L, about 22 μg/L, about 23 μg/L, about 24 μg/L, about 25 μg/L, about 26 μg/L, about 27 μg/L, about 28 μg/L, about 29 μg/L, about 30 μg/L, about 31 μg/L, about 32 μg/L, about 33 μg/L, about 34 μg/L, about 35 μg/L, about 36 μg/L, about 37 μg/L, about 38 μg/L, about 39 μg/L, about 40 μg/L, about 41 μg/L, about 42 μg/L, about 43 μg/L, about 44 μg/L, about 45 μg/L, about 46 μg/L, about 47 μg/L, about 48 μg/L, about 49 μg/L, or about 50 μg/L.

In some aspects, the steady-state plasma concentration is maintained for about 7 days. In some aspects, the steady-state plasma concentration is maintained for about 3-10 days. In some aspects, the steady-state plasma concentration is maintained for about 3 days, about 4 days, about 5 days, about 6 days, about 7 days, about 8 days, about 9 days, or about 10 days.

In some aspects, the initial burst of apomorphine is about 5% of the total dose of apomorphine or a pharmaceutically acceptable salt thereof. In some aspects, the initial burst of apomorphine is about 1-50% of the total dose of apomorphine or a pharmaceutically acceptable salt thereof. In some aspects, the initial burst of apomorphine is about 30-50% of the total dose of apomorphine or a pharmaceutically acceptable salt thereof. In some aspects, the initial burst of apomorphine is about 10-30% of the total dose of apomorphine or a pharmaceutically acceptable salt thereof. In some aspects, the initial burst of apomorphine is about 1-15% of the total dose of apomorphine or a pharmaceutically acceptable salt thereof. In some aspects, the initial burst of apomorphine is about 1%, about 2%, about 3%, about 4%, about 5%, about 6%, about 7%, about 8%, about 9%, about 10%, about 11%, about 12%, about 13%, about 14%, or about 15% of the total dose of apomorphine or a pharmaceutically acceptable salt thereof.

In some aspects, following the initial burst of apomorphine or a pharmaceutically acceptable salt thereof, there is an extended release of apomorphine for about 7 days. In some aspects, the extended release corresponds to a release of about 5% to about 25% release of the total administered dose of apomorphine or a pharmaceutically acceptable salt thereof per day. In some aspects, the extended release corresponds to a release of about 10% to about 15% release of the total administered dose of apomorphine or a pharmaceutically acceptable salt thereof per day.

In some aspects, the initial burst of apomorphine is completed about 0.5 to about 24 hours after injection. In some aspects, the initial burst of apomorphine is completed about 1 to about 12 hours after injection. In some aspects, the initial burst of apomorphine is completed about 0.5 hours, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after injection.

In some aspects, following the initial burst of apomorphine or a pharmaceutically acceptable salt thereof, there is a zero-order or first-order release profile. In some aspects, there is a zero-order release profile. In some aspects, there is a first-order release profile.

In some aspects, the zero-order or first-order release profile persists for about 2, about 3, about 4, about 5, about 6, about 7, about 8, about 9, about 10, about 11, about 12, about 13, or about 14 days. In some aspects, the zero-order or first-order release profile persists for about 5-10 days.

In some aspects, the zero-order release profile corresponds to a release of about 12% to about 14% release of the total administered dose of apomorphine or a pharmaceutically acceptable salt thereof per day. In some aspects, the zero-order release profile corresponds to a release of about 5% to about 14% release of the total administered dose of apomorphine or a pharmaceutically acceptable salt thereof per day. In some aspects, the zero-order release profile corresponds to a release of about 5%, about 6%, about 7%, about 8%, about 9%, about 10%, about 11%, about 12%, about 13%, or about 14% release of the total administered dose of apomorphine or a pharmaceutically acceptable salt thereof per day.

In some aspects, the elimination half-life of apomorphine is about 40 minutes to about 60 minutes. In some aspects, the elimination half-life of apomorphine is about 20 minutes to about 120 minutes. In some aspects, the elimination half-life of apomorphine is about 20 minutes, about 30 minutes, about 40 minutes, about 50 minutes, about 60 minutes, about 70 minutes, about 80 minutes, about 90 minutes, about 110 minutes, or about 120 minutes.

In some aspects, the steady state plasma profile of apomorphine from day 1 to day 7 following administration exhibits a mean Cₘₐₓ value no greater than the steady state plasma level of apomorphine provided by 2 mg of immediate release subcutaneous injection of apomorphine hydrochloride.

In some aspects, administration of the stable pharmaceutically acceptable formulation results in less hypotension relative to administration with immediate release subcutaneous apomorphine hydrochloride injection.

In some aspects, administration of the stable pharmaceutically acceptable formulation results in less nausea relative to administration with immediate release subcutaneous apomorphine hydrochloride injection.

In some aspects, administration of the stable pharmaceutically acceptable formulation results in less adverse injection site reactions relative to administration with immediate release subcutaneous apomorphine hydrochloride injection.

### Kit

In some aspects, the present disclosure provides a kit. In a further aspect, the kit comprises a first vial comprising a concentrated form of the stable pharmaceutically acceptable formulations described herein or one of the long-acting dosage forms described herein. In a further aspect, the kit comprises a second vial comprising a pharmaceutically acceptable diluent. In a further aspect, the kit comprises a first syringe suitable for withdrawing the pharmaceutically acceptable diluent from the second vial. In a further aspect, the kit comprises an adapter, which can operably attach to the first syringe and is suitable for dispensing the pharmaceutically acceptable diluent into the first vial. In a further aspect, the kit comprises a second syringe suitable for withdrawing a liquid from the second vial and for injecting the liquid into a subject. In a further aspect, the kit comprises instructions for diluting the concentrated form and for administering the stable pharmaceutically acceptable formulation or the long-acting dosage form to a patient in need thereof.

### Conditions to be Treated

The present disclosure relates in part to the acute, intermittent treatment of hypomobility, “off” episodes (“end-of-dose wearing off” and unpredictable “on/off” episodes) associated with advanced Parkinson's disease.

### EXAMPLES

The following are non-limiting, illustrative examples, and are not intended to limit the scope of the claims. All references disclosed in the Examples are incorporated by reference in their entirety.

### Example 1: Method of Preparing Formulations of Apomorphine Free Base or Pharmaceutically Acceptable Salt Thereof

A general procedure of preparing a formulation of the present disclosure is provided as follows.

Water for Injection is first placed in a suitable container.

Excipients are added to the WFI and mixed until they are dissolved completely.

Optionally, a co-solvent is added and the solution is stirred until a clear solution is obtained.

The pH of the solution is adjusted to the desired range.

API is added, and the solution is stirred until API dissolves completely.

The pH of the solution is adjusted to the desired pH, as needed.

### Example 2: Long-Acting Release Profile Simulation

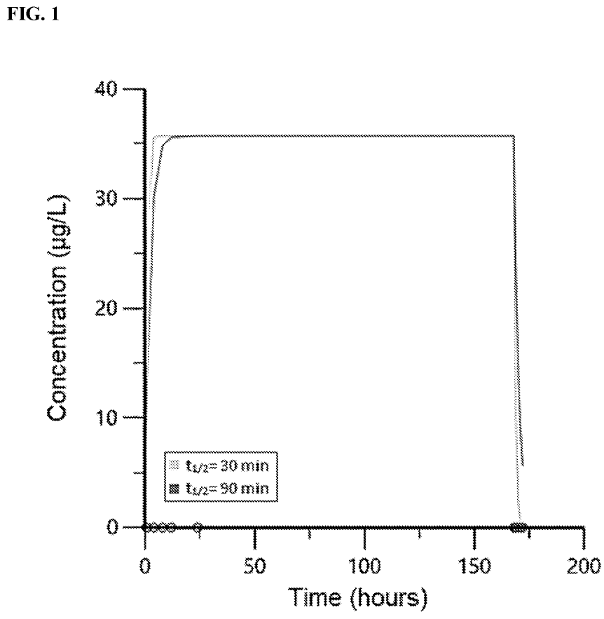

A one-compartment model simulation of apomorphine release was performed using a dosing rate of 10 mg/hr and a clearance value of 280 L/hr. Two simulations were carried out, with a half-life of either 30 minutes or 90 minutes. With either half-life, the time taken to reach steady state is similar, and expected steady state concentration of apomorphine is about 35 μg/L (Table 1, FIG. 1).

TABLE 1

| | |
|---|---|
| T_{1/2} = 30 min | T_{1/2} = 90 min |

| Time | Concentration | Time | Concentration |
|---|---|---|---|
| (hours) | (ug/L) | (hours) | (ug/L) |
| 0 | 0 | 0 | 0 |
| 0.5 | 17.9 | 0.5 | 7.4 |
| 1 | 26.8 | 1 | 13.2 |
| 4 | 35.6 | 4 | 30.1 |
| 8 | 35.7 | 8 | 34.8 |
| 12 | 35.7 | 12 | 35.6 |
| 24 | 35.7 | 24 | 35.7 |
| 168 | 35.7 | 168 | 35.7 |
| 168.5 | 17.9 | 168.5 | 28.3 |
| 169 | 8.9 | 169 | 22.5 |
| 170 | 2.2 | 170 | 14.2 |
| 171 | 0.6 | 171 | 8.9 |
| 172 | 0.1 | 172 | 5.6 |

### Example 3: Synthesis of Multi-Block Copolymers

Previous reports of apomorphine-loaded polymeric compositions demonstrated a low encapsulation efficiency and active load of apomorphine, likely resulting at least in part from oxidation of apomorphine. Regnier-Delplace, C., et al. 2013. PLGA microparticles with zero-order release of the labile anti-Parkinson drug apomorphine. International Journal of Pharmaceutics. 443:68-79. Accordingly, apomorphine-containing polymeric compositions were prepared under various conditions and analyzed for improved drug load of apomorphine.

This example describes the synthesis and characterization of multi-block copolymers used in the preparation of apomorphine-loaded microspheres.

[Poly(D,L-Lactide)-co-poly(ethylene glycol)-co-poly(D,L-lactide)]-b-[poly(glycolide-co-L-lactide)] multi-block copolymers with 10/90 and 20/80 block ratio were synthesized using similar procedures as described in WO 2013/015685. In brief, poly(DL-Lactide)-co-PEG1000-co-poly(DL-Lactide) pre-polymer with Mₙ of around 2000 g/mol (abbreviated as “PDLA-PEG1000-PDLA” or “LP10L20”) was prepared by ring-opening polymerisation of D,L-Lactide using poly(ethylene glycol) (“PEG”) with a molecular weight 1000 g/mol (abbreviated as “PEG1000”) as initiator and stannous octanoate as a catalyst. Poly(glycolide-co-L-lactide) pre-polymers with Mₙ of about 4000 g/mol (abbreviated as “PGLLA” or “GLL40”) were synthesised by solution ring-opening polymerisation of glycolide and L-lactide with a 15/85 monomer ratio in p-dioxane using 1,4-butanediol as initiator and stannous octanoate as a catalyst. Molecular weight and chemical structure of the pre-polymers were analysed by ¹H-NMR.

[PDLA-PEG1000-PDLA]-b-[PGLLA] multi-block copolymers with a block ratio of 10/90 or 20/80 w/w were prepared by chain-extension of the PDLA-PEG1000-PDLA pre-polymer with the PGLLA pre-polymer in p-dioxane using 1,4-butanediisocyanate as a chain extender followed by freeze-drying to remove p-dioxane. The polymers are abbreviated as “10LP10L20-GLL40” and “20LP10L20-GLL40”.

A [poly(ε-caprolactone)-co-poly(ethylene glycol)-co-poly(ε-caprolactone)]-b-[poly(D,L-lactide)] multi-block copolymer with a block ratio of 5/95 (abbreviated as “5CP30C40-L40”) was synthesized using similar procedures as described above. 5CP30C40-L40 is a multi-block copolymer composed of a [PCL-PEG3000-PCL]pre-polymer segment (A) with a molecular weight of 4000 g/mole (containing 75 mole % of polyethylene glycol with a molecular weight of 3000 g/mole) and an amorphous poly(DL-Lactide) pre-polymer segment (B) with a molecular weight of 4000 g/mole which are chain extended in a 5/95 wt. % block ratio by 1,4-butanediisocyanate.

A [poly(D,L-Lactide)-co-poly(ethylene glycol)-co-poly(D,L-lactide)]-b-[poly(p-dioxanone)] multi-block copolymer with a block ratio of 60/40 (abbreviated as “60LP2L20-D27”) was synthesized using similar procedures as described in WO 2020/071912. 60LP2L20-D27 is a multi-block copolymer composed of a [poly(D,L-lactide)-co-poly(ethylene glycol)-co-poly(D,L-lactide)] [PDLA-PEG200-PDLA] pre-polymer segment (A) with a molecular weight of 2000 g/mole (containing 10 mole % of polyethylene glycol with a molecular weight of 200 g/mole) and a semi-crystalline poly(p-dioxanone) pre-polymer segment (B) with a molecular weight of 2700 g/mole which are chain extended in a 60/40 wt. % block ratio by 1,4-butanediisocyanate

All polymers were analysed for their chemical composition, intrinsic viscosity, residual p-dioxane content, and thermal characteristics.

The chemical composition (monomer ratio) and molecular weight (MW) of the pre-polymers, as well as the block ratio of the multi-block copolymers was determined by ¹H-NMR. For this determination a Bruker Avance DRX 500 MHz NMR spectrometer B AV-500 was used equipped with Bruker Automatic Sample Changer BACS 60 (VARIAN®) operating at 500 MHz. The di delay time was set to 20 s, and the number of scans was 16. Spectra were recorded from 0 to 14 ppm. ¹H-NMR samples were prepared by adding about 1.3 g of deuterated chloroform to about 25 mg of polymer.

Intrinsic viscosity was measured using an Ubbelohde Viscosimeter (DIN), type OC, SI ANALYTICS® The measurements were performed in chloroform at 25° C. The temperature was controlled using a water bath. The polymer concentration in chloroform was such that the relative viscosity was in the range of 1.2-2.0.

Residual p-Dioxane content was determined using a gas chromatography flame ionization detection (“GC-FID”) headspace method. Measurements were performed on a GC-FID Combi Sampler supplied with an AGILENT® Column, DB-624/30 m/0.53 mm. Samples were prepared in dimethylsulphoxide (“DMSO”). Residual solvent content was determined using p-dioxane calibration standards.

Modulated differential scanning calorimetry (“MDSC”) was used to determine the thermal behaviour of the multi-block copolymers using a Q2000 MDSC (TA INSTRUMENTS®, Ghent, Belgium). About 5-10 mg of dry material was accurately weighed and heated under a nitrogen atmosphere from −85° C. to 180° C. at a heating rate of 2° C./min and a modulation amplitude of +/−0.42° C. every 80 seconds. The glass transition temperature (T_{g}, midpoint), melting temperature (maximum of endothermic peak, Tₘ) were determined from the reversing heat flow of the first heating run, whereas the melting enthalpy (ΔHₘ), was calculated from the sum of the surface areas of the melting endotherms of the reversing and non-reversing heat flow of the first heating run. Temperature and enthalpy were calibrated using an indium standard.

Table 2 lists the characteristics of the multi-block copolymers.

TABLE 2
Characteristics of multi-block copolymers usedfor
the preparation of apomorphine-loaded microspheres

| | | Block | | | Residual | | | |
|---|---|---|---|---|---|---|---|---|
| | | ratio | PEG MW | IV | dioxane | Tg | Tₘ | ΔHₘ |
| RCP | Polymer grade | (w/w) | (g/mol) | (dl/g) | (ppm) | (° C.) | (° C.) | (J/g) |
| 2021 | 10LP10L20-GLL40 | 10/90 | 1000 | 0.85 | <20 | 44 | 103 | 2.7 |
| 1919A | 20LP10L20-GLL40 | 20/80 | 1000 | 0.85 | 200 | 35 | — | — |
| 1926 | 60LP2L20-D27 | 60/40 | 200 | 0.61 | 533 | 2.7 | 85 | 50 |
| 1668 | 5CP30C40-L40 | 5/95 | 3000 | 0.70 | <30 | 42 | — | — |

### Example 4: Solubility of APO-HCL in Various Solvents

Poorly water soluble compounds are preferably encapsulated via oil-in-water (“O/W”) single emulsion extraction/evaporation microencapsulation in which the compound and polymer are co-dissolved in the oil phase. To prepare microspheres with a loading of 30-50 wt. %, the solubility of the compound in the oil phase should preferably be around 100 mg/mL. Dichloromethane (“DCM”) is the preferred solvent for use in the oil phase, and was used accordingly in Regnier-Delplace, et al., supra. However, apomorphine hydrochloride hemihydrate (“APO-HCL”, molecular weight 313 g/mole) was found to be very poorly soluble in dichloromethane alone (<0.5 mg/mL). Also, APO-HCL solubility in other solvents known to be suitable for O/W microencapsulation, such as ethyl acetate and chloroform, was too low (Table 3).

TABLE 3
Solubility of APO-HCL in organic solvents
typically used in O/W microencapsulation

| | | Actual concentration |
|---|---|---|
| Solvent | Experiment code | (mg/ml) |
| DCM | DW-2001-006-04 | <0.5 mg/ml |
| Ethyl Acetate | DW-2001-006-11 | <0.4 mg/ml |
| Chloroform | DW-2001-006-13 | <18.5 mg/ml |

Because of the low solubility of apomorphine in dichloromethane, ethyl acetate and chloroform, the use of a co-solvent system was explored. For a co-solvent mixture it is important that the co-solvent is compatible with DCM and that, to enable the preparation of apomorphine-loaded microspheres with a target loading of 30-50 wt. %, the solubility of APO-HCL in the co-solvent is significantly higher than 100 mg/mL, such as ≥200 mg/mL. The solubility of APO-HCL was tested in various other organic solvents potentially suitable for use in a co-solvent system with DCM.

The solubility of APO-HCL in the majority of the tested solvents tested was very low (<20 mg/mL) (Table 3). The solubility of APO-HCL in benzyl alcohol (51 mg/ml) and methanol (92.8 mg/ml) was higher. However, the color of the solutions turned into dark brown (benzyl alcohol) or green (methanol) indicating severe oxidation of APO-HCL in these solvents. Dimethyl sulfoxide (“DMSO”), N-methyl pyrrolidone (“NMP”) and dimethyl formamide (“DMF”) appeared to exhibit the required characteristics. The solubility of APO-HCL in DMSO (513.6 mg/ml), DMF (507.5 mg/ml) and NMP (539.5 mg/ml) was sufficient to allow use of these solvents in a co-solvent system with DCM, and surprisingly only a very slight change in the color of the solutions (slightly yellowish) was observed, indicating that oxidation of apomorphine hardly occurred in these solvents.

TABLE 4
Solubility of APO-HCL in various solvents for use
in a co-solvent mixture with dichloromethane

| | | Maximum concentration | |
|---|---|---|---|
| Solvent | Experiment code | (mg/ml) | |
| Acetic acid | DW-2001-006-08 | <2.7 | mg/ml |
| Acetone | DW-2001-006-09 | <0.4 | mg/ml |
| Acetonitrile | DW-2001-052-07 | <12.4 | mg/ml |
| Benzyl alcohol | DW-2001-006-05 | 51.0 | mg/ml |
| Dioxane | DW-2001-052-05 | <12.4 | mg/ml |
| Diethyl ether | DW-2001-052-08 | <13.7 | mg/ml |
| DMF | DW-2001-052-11 | 507.5 | mg/ml |
| DMSO | DW-2001-006-12 | 513.6 | mg/ml |
| Ethanol | DW-2001-052-09 | <12.7 | mg/ml |
| Ethyl Formate | DW-2001-006-10 | <0.6 | mg/ml |
| IPA | DW-2001-052-03 | <12.7 | mg/mL |
| NMP | DW-2001-052-10 | 539.5 | mg/ml |
| Methanol | DW-2001-006-06 | 92.8 | mg/ml |
| THF | DW-2001-052-01 | <13.3 | mg/ml |
| Toluene | DW-2001-052-06 | <12.3 | mg/ml |

Based on the high solubility and the limited oxidation of APO-HCL in DMSO, and the United States Food and Drug Administration's Generally Recognized as Safe status of DMSO (class III solvent), DMSO was selected for its suitability to be used in a co-solvent system with DCM. However, given the solubility also observed in benzyl alcohol, DMF, NMP, and methanol, these are also within the scope of the present disclosure, and the use of DMSO herein is not intended to be limiting.

Since DMSO is highly miscible with water, the volume fraction of DMSO to be used in a co-solvent system with DCM should not be too high as to assure proper droplet formation, particle hardening and acceptable encapsulation efficiency. On the other hand, DCM/DMSO co-solvent systems with only 10-20% DMSO had insufficient APO-HCL solubility for the preparation of apomorphine-loaded microspheres with high apomorphine loading. DCM/DMSO co-solvent systems containing 25 to 35 vol. % DMSO were found to be most optimal as it allowed proper emulsification and microparticle formation as well as microencapsulation of apomorphine at high target loading and encapsulation efficiency.

### Solubility of APO-HCL in the Continuous Phase

The aqueous solubility of APO-HCL was found to be 17.8 mg/ml (20° C.). To prevent loss of APO from the microsphere (“MSP”) during microencapsulation and solvent extraction, the use of a polyvinyl alcohol (“PVA”) based aqueous extraction medium with significantly lower APO-HCl solubility is preferred. The solubility of APO-HCl was tested in different aqueous PVA solutions. Significantly lower solubility of APO-HCl were obtained for 0.4% PVA, 5% NaCl (<0.3 mg/mL), 0.4% PVA, 100 mM Tris, pH 8.5 (<<1 mg/mL) and 0.5% PVA, 50 mM PB, pH 8 (<<1 mg/mL). The color of both 0.4% PVA solutions that contained the Tris buffer and the phosphate buffer turned dark brown after adding APO-HCL, due to severe oxidation of apomorphine. The 0.4% PVA solution containing 5% NaCl showed only very limited discoloration after adding APO-HCL. Therefore, the 0.4% PVA solution containing 5% NaCl was selected for use as the continuous phase for the manufacturing of the apomorphine loaded microspheres.

TABLE 5
Solubility of APO-HCL in aqueous media

| | Maximum concentration | |
|---|---|---|
| Solution | (mg/ml) | |
| Water | 17.9 | mg/ml |
| 0.4% PVA, 5% NaCl | <0.3 | mg/mL |
| 0.4% PVA, 100 mM Tris, pH 8.5 | <<1 | mg/mL |
| 0.5% PVA, 50 mM PB, pH 8 | <<1 | mg/mL |

### Example 5: Preparation and Characterization of Apomorphine Loaded Microspheres Composed of 20LP10L20-GLL40 (Round 1)

Apomorphine loaded microspheres with a target loading of approximately 42.0% apomorphine free base were prepared of 20LP10L20-GLL40 via oil-in-water (“O/W”) membrane emulsification followed by solvent extraction/evaporation.

First a 4.0 wt. % PVA solutions was prepared by dissolving 40 g of PVA with a viscosity of 4.3-5.7 mPa s (40 g/L; water) and a degree of hydrolysis (USP) of 85-89% (PVA 5-88 EMPROVE® ESSENTIAL, Merck KGaA) in 960 g of ultrapure (UP)-water heated at 75° C., followed by filtration of the solution over a 5 μm filter at room temperature. A 0.4 wt. % PVA solution with 5% NaCl was obtained by diluting 500 g of the 4 wt % PVA solution with 4250 g UP water and adding 250 g of NaCl under stirring. After complete dissolution of NaCl, the resulting solution was filtered over 0.2 μm polyethersulfone (“PES”) filter capsule.

A 0.05 wt % Tween-80 solution was prepared by adding 2.5 g of Tween into 5 L of UP water and stirring the solution for 15 minutes at room temperature.

Then 0.4 g APO-HCL was dissolved in DMSO to a concentration of 500 mg/mL whereafter DCM was added to a DMSO/DCM ratio of 20/80% v/v. Finally 0.4 g polymer was added yielding a DMSO/DCM 20/80 solution containing 7.2% polymer and 7.0% APO-HCL. The solution—or dispersed phase (“DP”)—was stirred overnight to assure complete dissolution of polymer and APO-HCL.

Following filtration over a 0.2 μm polytetrafluoroethylene (“PTFE”) filter, DP was emulsified with an aqueous 0.4 wt. % PVA 5-88 5% NaCl based continuous phase (“CP”) by pumping DP at a controlled flow rate of 1.3 mL/min via a membrane with 20 μm pores into a stirred vessel into which CP was pumped at a CP/DP ratio of 100 v/v. The formed O/W emulsion was stirred at 200 rpm for 2 hours at room temperature followed by 1-2 hours under elevated temperature (40° C.) under an airflow of 5 L/min to extract and evaporate DCM and harden the microspheres. After completion of the solvent extraction process, the hardened microspheres were collected by filtration and washed three times with 250 mL 0.05 wt. % Tween-80 solution and three times with 250 mL WFI. Finally the microspheres were transferred to 10 mL lyophilization vials, frozen at −70° C. and lyophilized according to the program detailed in Table 6.

TABLE 6
Lyophilization cycle used for vacuum drying
of apomorphine loaded microspheres

| Step | | Time | Shelf | Vacuum |
|---|---|---|---|---|
| # | Phase | (hh:mm) | Temp | (mbar) |
| 1 | Loading | — | −45 | — |
| 2 | Freezing | 01:00 | −45 | — |
| 3 | Main drying | 01:00 | −10 | 2.0 |
| 4 | Main drying | 01:00 | −10 | 2.0 |
| 5 | Main drying | 08:00 | 20 | 2.0 |
| 6 | Main drying | 01:00 | 20 | 1.0 |
| 7 | Final drying | 03:00 | 40 | 0.01 |
| 8 | Final drying | 03:00 | 40 | 0.01 |
| 9 | Final drying | 01:00 | 20 | 0.01 |
| 10 | Storage | — | 5 | 0.05 |

The resulting apomorphine loaded microspheres were analysed for their particle size distribution (“PSD”) using a HORIBA® LA-960 Laser Particle Size Analyser.

Characterization of microsphere surface morphology and particle shape was performed by scanning electron microscopy (“SEM”) using a JEOL® JCM-5000 NEOSCOPE™.

Apomorphine free base content of the microspheres was determined by elemental analysis. In brief, 2.5-5 mg of apomorphine loaded microspheres, APO-HCL and polymer were accurately weighed in a tin foil and combusted at 1150° C. in an ELEMENTAR® Micro Cube with an excess of oxygen to ensure complete sample combustion. The formed N₂, CO₂, H₂O and SO₂ gasses were retained by an adsorption column and eluted separately and analysed using a thermal conductivity detector. By comparing the nitrogen content of the apomorphine loaded microspheres with that of APO-HCL and polymer, the apomorphine free base content of the apomorphine loaded microspheres was calculated.

The in vitro release kinetics of the prepared apomorphine microspheres was determined by incubating 10 mg of apomorphine microsphere in 15 mL polypropylene centrifuge tube containing 10 ml in vitro release buffer (100 mM PO₄ buffer, 0.5% Tween-20, 0.02% NaN₃, pH 7.4 and 0.15% sodium ascorbate and 0.15% sodium metabisulfite as anti-oxidants) which were placed in a climate chamber thermostated at 37° C. At predetermined time points (2 and 6 hours, 1, 2, 4 and 7 days), following centrifugation of the vials, 9.0 ml of the supernatant was collected and replaced by 9.0 mL of fresh buffer. The supernatant was stored at 2-8° C., until analysis. Apomorphine free base concentrations in the release buffer were determined via reversed phase ultra performance liquid chromatography (“UPLC”) with UV-detection using a Waters Acquity H-Class UPLC system, equipped with a PDA or UV detector, an Acquity BEH C18 column (50×2.1 mm, 1.7 μm), maintained at 40° C. Mobile phase A consisted of a 20 mM phosphate buffer pH 2.5 and acetonitrile was used as mobile phase B. The mobile phase composition started at 5% B and increased to 35% B in 1.5 minutes, at a constant flow rate of about 0.6 mL/min. Detection was performed at 273 nm.

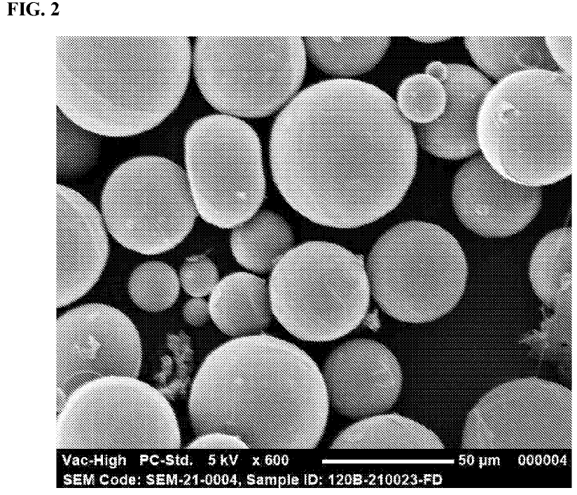

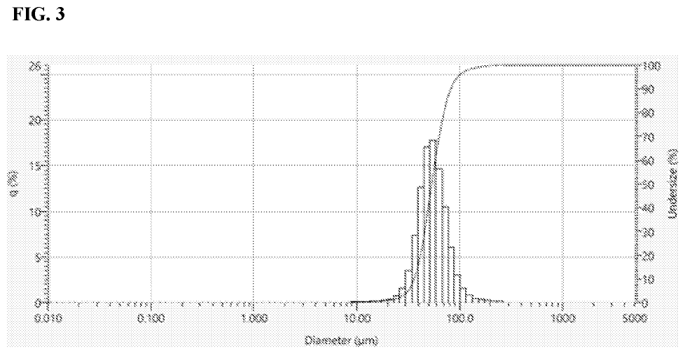

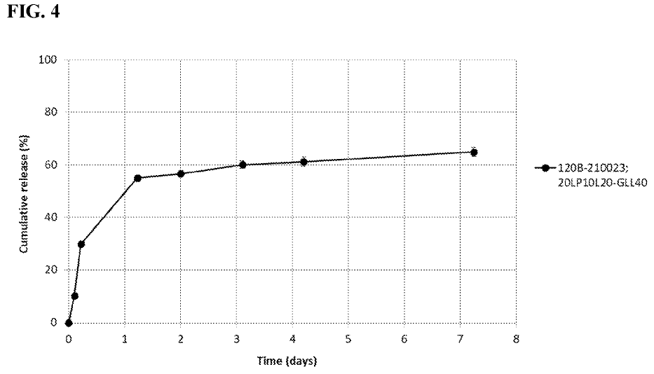

Scanning electron microscopy shows that the O/W microencapsulation process yielded spherical microspheres with a smooth surface without any pores (FIG. 2) and a narrow particle size distribution with a median particle size (“D50”) of 54 μm and a coefficient of variance (“CV”) of 41%. FIG. 3 shows the particle size distribution of 120B-210023). The apomorphine free base content of the 20LP10L20-GLL40 microspheres was 30.2 wt. % representing an encapsulation efficiency (“EE”) of 71.9%. Apomorphine was released in vitro relatively fast with more than 50% released per day followed by slow release thereafter. After 7 days, around 65% of the encapsulated apomorphine had been released (FIG. 4).

### Example 6: Preparation of Apomorphine Loaded Microspheres (Round 2)

Apomorphine loaded microspheres were prepared of various polymers according to the O/W membrane emulsification procedure described in Example 5. To overcome the APO-HCL solubility and precipitation issues encountered in Example 5, a 25/75% v/v DMSO/DCM co-solvent system was used with increased APO-HCL solubility and the procedure for preparation of DP was adjusted. In brief, 0.4 g APO-HCL was dissolved in DMSO to a concentration of 500 mg/mL, and 0.4 g polymer was dissolved in 1 mL dichloromethane. The polymer solution was added to the APO-HCL solution and the combined solution was stirred overnight resulting in a clear DMSO/DCM 25/75% v/v solution containing 5.4-7.3 wt. % polymer and 8.0-8.6 wt. % APO-HCL (Table 7).

The solutions were stirred overnight to ensure complete dissolution of polymer and APO-HCl. Following filtration over a 0.2 μm PTFE filter, DP was emulsified with an aqueous 0.4 wt. % PVA 5-88 5% NaCl based CP by pumping DP at a flow rate of 1.3 mL/min via a membrane with 20 μm pores into a stirred vessel into which CP solution was pumped at a CP/DP ratio of 100 v/v. The O/W emulsion was stirred at 200 rpm for 2 hours at room temperature followed by 1-2 hours under elevated temperature (40° C.) under an airflow of 5 L/min to extract and evaporate DCM and harden the microspheres. After completion of solvent evaporation, the microspheres were collected by filtration, washed with 0.05 wt. % aqueous Tween-80 solution and WFI and dried by lyophilization as described in Example 5.

TABLE 7
Formulation and process settings used for preparation of apomorphine loaded microspheres

| | | Oil phase | | Target | | | | |
|---|---|---|---|---|---|---|---|---|
| | | Pol. | APO-HCl | APO free | | | | Stir. |
| 120B | | conc. | conc | base load | CP/DP | DP Inj | CP Inj | Speed |
| batch | Polymer grade | (wt. %) | (wt. %) | (wt %) | (v/v) | (mL/min) | (ml/min) | (rpm) |
| 210036 | 60LP2L20-D27 | 7.2 | 8.2 | 45.4% | 100 | 1.3 | 130 | 900 |
| 210037 | 20LP10L20-GLL40 | 5.4 | 8.0 | 51.0% | 100 | 1.3 | 130 | 900 |
| 210038 | 5CP30C40-L40 | 5.9 | 8.6 | 50.7% | 100 | 1.3 | 130 | 900 |

In contrast to what was observed during the preparation of the apomorphine-loaded microspheres prepared in Example 5, no precipitation of apomorphine was observed when using a 25/75% v/v DMSO/DCM based DP for the preparation of the apomorphine-loaded microparticles. However, extensive discoloration was observed. The 60LP2L20-D27 based apomorphine-loaded microspheres turned completely green during solvent extraction and microparticle hardening. 20LP10L20-GLL40 based apomorphine-loaded microspheres were slightly greenish. 5CP30C40-L40 based apomorphine-loaded microspheres did not show any discoloration and were white after lyophilization, but turned green during in vitro release testing.

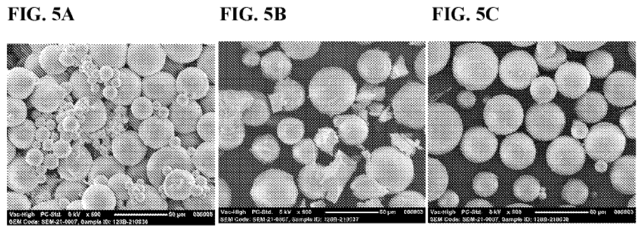

The resulting microspheres were analysed according to the methods described in Example 5. Scanning electron microscopy shows that the O/W microencapsulation process yielded spherical microspheres with a smooth surface without any pores (FIGS. 5A-5C). The 60LP2L20-D27 based apomorphine-loaded microspheres (batch 120B-210036) showed extensive agglomeration due to which the particle size distribution could not be determined. The 5CP30C40-L40 (batch 120B-210038) and 20LP10L20-GLL40 based apomorphine loaded microspheres (batch 120B-210037) had a D50 median particle size close to 50 μm and a CV of around 34%. The apomorphine content of the 60LP2L20-D27 based apomorphine-loaded microspheres (batch 120B-210036) was only 3.2% representing an encapsulation efficiency (EE) of only 7.1%. The low encapsulation efficiency was attributed to poor and/or slow crystallization of the semi-crystalline poly(dioxanone)-based D27 block of 60LP2L20-D27. The apomorphine content of apomorphine loaded microspheres prepared of the amorphous 20LP10L20-GLL40 and 5CP30C40-L40 multi-block copolymers was 37-39%, a drastically improved apomorphine loading representing an EE of approximately 75%.

TABLE 8
Characteristics of apomorphine-loaded microspheres

| | | | | | Residual | | |
|---|---|---|---|---|---|---|---|
| | | PEG | PSD Apomorphine MSP | | DCM | Apomorphine | |
| 120B | | content | D 50 | CV | content | free base load | EE |
| batch | Polymer grade | (mole %) | (μm) | (%) | (ppm) | (wt %) | (%) |
| 210036 | 60LP2L20-D27 | 6% | —* | —* | ND | 3.2% | 7.1% |
| 210037 | 20LP10L20-GLL40 | 10% | 49 | 33% | ND | 38.9% | 76.3% |
| 210038 | 5CP30C40-L40 | 4% | 48 | 35% | ND | 37.1% | 73.2% |

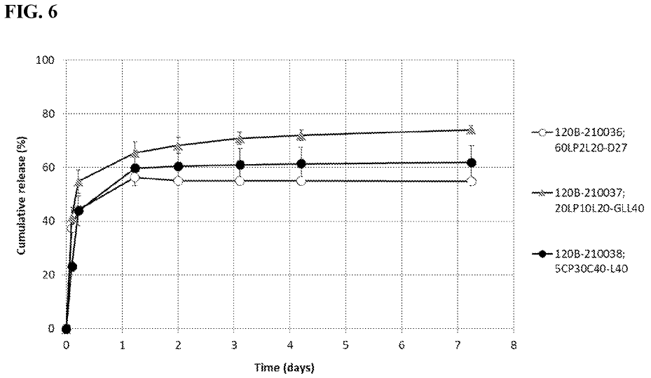

FIG. 6 shows the cumulative in vitro release of apomorphine. All batches, irrespective of polymer composition showed similar in vitro release kinetics with a high burst release varying from 40 to 66% and hardly any further release thereafter. The 60LP2L20-D25 and 5CP30C40-L40 based formulations did not show any further release after day 1, whereas the 20LP10L20-GLL40 based apomorphine microspheres showed slow but gradual release between day 1 and day 7, similar to the 20LP10L20-GLL40 based apomorphine microspheres of Example 5.

### Example 7: Preparation of Apomorphine Loaded Microspheres (Round 3)

Apomorphine loaded microspheres were prepared using the same polymers and according to the same O/W membrane emulsification procedure described in Example 6 except that 0.15% sodium ascorbate and 0.15% sodium metabisulfite were added as antioxidants to the continuous phase to prevent oxidation and discoloration of apomorphine during solvent extraction and microparticle hardening. In brief, 0.8 g APO-HCL was dissolved in DMSO to a concentration of 400 mg/mL, and the polymer was dissolved in dichloromethane to a concentration of 133 mg/mL. The polymer solution was added to the APO-HCL solution and the combined solution was stirred overnight resulting in a clear DMSO/DCM 25/75% v/v solution containing ˜7 wt. % polymer and ˜8.0 wt. % APO-HCL (Table 9). The solutions were stirred overnight to assure complete dissolution of polymer and APO-HCL. Following filtration over a 0.2 μm PTFE filter, DP was emulsified with an aqueous 0.4 wt. % PVA 5-88 5% NaCl based CP containing 0.15% sodium ascorbate and 0.15% sodium metabisulfite by pumping DP at a flow rate of 1.3 mL/min via a membrane with 20 μm pores into a stirred vessel into which CP solution was pumped at a CP/DP ratio of 100 v/v. The O/W emulsion was stirred at 200 rpm for 2 hours at room temperature followed by 1-2 hours under elevated temperature (40° C.) under an airflow of 5 L/min to extract and evaporate DCM and harden the microspheres. After completion of solvent evaporation, the microspheres were collected by filtration, washed with 0.05 wt. % aqueous Tween-80 solution and WFI and dried by lyophilization as described in example 5.

TABLE 9
Formulation and process settings used for preparation of apomorphine loaded microspheres

| | | Oil phase | | Target | | | | |
|---|---|---|---|---|---|---|---|---|
| | | Pol. | APO-HCl | APO free | | | | Stir. |
| 120B | | conc. | conc | base load | CP/DP | DP Inj | CP Inj | Speed |
| batch | Polymer grade | (wt. %) | (wt. %) | (wt %) | (v/v) | (mL/min) | (ml/min) | (rpm) |
| 210051 | 60LP2L20-D27 | 7.2 | 7.7 | 44.2% | 100 | 1.3 | 130 | 900 |
| 210052 | 20LP10L20-GLL40 | 7.1 | 8.2 | 45.7% | 100 | 1.3 | 130 | 900 |
| 210053 | 5CP30C40-L40 | 7.2 | 8.2 | 45.6% | 100 | 1.3 | 130 | 900 |

The resulting microspheres were analysed according to the methods described in Example 5. The microspheres were spherical and had a smooth surface without any pores. The 60LP2L20-D27 based apomorphine-loaded microspheres (batch 120B-210051) showed extensive agglomeration due to which the particle size distribution could not be determined. The 5CP30C40-L40 (batch 120B-210053) and 20LP10L20-GLL40 based apomorphine loaded microspheres (batch 120B-210052) had a D50 median particle size close to 50 μm and a CV of 27-30%. The apomorphine content of 120B-210036 was only 3.9% representing an EE of only 8.8%. The apomorphine content of 120B-210052 and 120B-210053 were much higher, at 37.5% (EE 82.1%) and 33.6 (73.6%), respectively.

Residual dichloromethane content of the microspheres was determined by gas chromatography with headspace injection and flame-ionization detection. In brief, 100 mg of sample was dissolved in 5.0 mL of DMSO containing octane as the internal standard. The samples were analyzed by GC-Headspace using an Agilent 6850 gas chromatograph, equipped with a Combi-Pal headspace sampler. The calibration range of the method was 18-4902 ppm DCM in 100 mg sample, using a first order linear regression (Weighing factor=1/X). The residual dichloromethane content of 60LP2L20-D27 based apomorphine-loaded microspheres (batch 120B-210051) was <55 ppm, 20LP10L20-GLL40 based apomorphine loaded microspheres had a residual DCM content of 399 ppm, whereas the 5CP30C40-L40-based apomorphine loaded microspheres had a residual DCM content of >5500 ppm.

Using a method similar to the method described in Example 3, the glass transition temperature (“Tg”) of the apomorphine-loaded microspheres was determined by modulated differential scanning calorimetry. About 5-10 mg of dry apomorphine loaded microspheres were accurately weighed and heated under a nitrogen atmosphere from −85° C. to 180° C. at a heating rate of 2° C./min and a modulation amplitude of +/−0.42° C. every 80 seconds, after which the sample was cooled and heated again using the same settings. The glass transition temperature (T_{g}, midpoint) was determined from the reversing heat flow of the first heating curve. Temperature and enthalpy were calibrated using an indium standard. The 60LP2L20-D27 based apomorphine-loaded microspheres (batch 120B-210051) had a Tg of only 6.8° C., whereas the 20LP10L20-GLL40 based apomorphine loaded microspheres (batch 120B-210052) had a glass transition above 40° C.

TABLE 10
Characteristics of apomorphine-loaded microspheres

| | | | | | Residual | | |
|---|---|---|---|---|---|---|---|
| | | PEG | PSD Apomorphine MSP | | DCM | Apomorphine | |
| 120B | | content | D 50 | CV | content | free base load | EE |
| batch | Polymer grade | (mole %) | (μm) | (%) | (ppm) | (wt %) | (%) |
| 210051 | 60LP2L20-D27 | 6% | —* | —* | <55 | 3.9% | 8.8% |
| 210052 | 20LP10L20-GLL40 | 10% | 50 | 30% | 399 | 37.5% | 82.1% |
| 210053 | 5CP30C40-L40 | 4% | 50 | 27% | >5500 | 33.6% | 73.6% |

*agglomerated

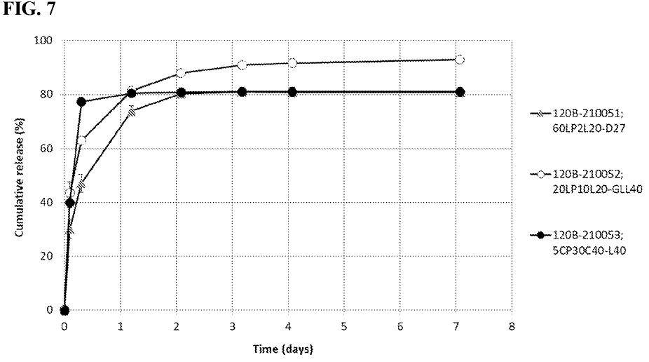

FIG. 7 shows the cumulative in vitro release of apomorphine from the batches that were prepared with antioxidants added to CP. Similar to the release profiles shown in Example 6, all batches, irrespective of polymer composition showed high burst and/or fast initial release of apomorphine followed by slow or hardly any release thereafter. However, the recovery, i.e. the total fraction of encapsulated apomorphine, was significantly higher as compared to the recovery obtained for the formulations prepared in example 6, which is attributed to the use of the antioxidants in CP.

### Example 8: Preparation of Slower Releasing Apomorphine Microspheres (Round 4)

To obtain apomorphine-loaded microspheres with extended release of apomorphine for approximately 1 week, apomorphine-loaded microspheres were prepared of 10LP10L20-GLL40 and of a 50/50% w/w blend of 10LP10L20-GLL40 and 20LP10L20-GLL40 using the O/W membrane emulsification procedure described in Example 7.

In brief, 0.8 g APO-HCL was dissolved in DMSO to a concentration of 400 mg/mL, and the polymer or the polymer blend was dissolved in dichloromethane to a concentration of 133 mg/mL. The polymer solution was added to the APO-HCL solution and the combined solution was stirred overnight resulting in a clear DMSO/DCM 25/75% v/v solution containing ˜7 wt. % polymer and ˜8.0 wt. % APO-HCL (Table 11).

TABLE 11
Formulation and process settings used for preparation of apomorphine loaded microspheres

| | | Oil phase | | Target | | | | |
|---|---|---|---|---|---|---|---|---|
| | | Pol. | APO-HCl | APO free | | | | Stir. |
| 120B | | conc. | conc | base load | CP/DP | DP Inj | CP Inj | Speed |
| batch | Polymer grade | (wt. %) | (wt. %) | (wt %) | (v/v) | (mL/min) | (ml/min) | (rpm) |
| 210105 | 10LP10L20-GLL40 | 7.2 | 8.2 | 45.4% | 100 | 1.3 | 130 | 900 |
| 210132 | 50/50 wt. % blend of | 7.2 | 8.0 | 45.1% | 100 | 1.3 | 130 | 900 |
| | 10LP10L20-GLL40 | | | | | | | |
| | 20LP10L20-GLL40 | | | | | | | |

The solutions were stirred overnight to assure complete dissolution of polymer and APO-HCl. Following filtration over a 0.2 μm PTFE filter, DP was emulsified with an aqueous 0.4 wt. % PVA 5-88 5% NaCl based CP containing 0.15% sodium ascorbate and 0.15% sodium metabisulfite by pumping DP at a flow rate of 1.3 mL/min via a membrane with 20 μm pores into a stirred vessel into which CP solution was pumped at a CP/DP ratio of 100 v/v. The O/W emulsion was stirred at 200 rpm for 2 hours at room temperature followed by 1-2 hours under elevated temperature (40° C.) under an airflow of 5 L/min to extract and evaporate DCM and harden the microspheres. After completion of solvent evaporation, the microspheres were collected by filtration, washed with 0.05 wt. % aqueous Tween-80 solution and WFI and dried by lyophilization as described in Example 5.

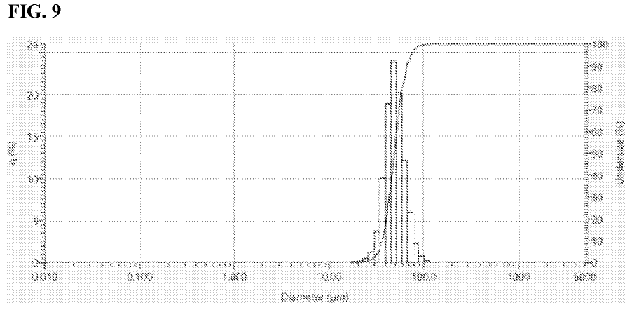

The resulting microspheres were analysed according to the methods described in Example 5. The microspheres were spherical and had a smooth surface without any pores. The 10LP10L20-GLL40 based apomorphine microparticles had a relatively large median particle size of 71 μm and a broad particle size distribution with a CV of 55%. The apomorphine microparticles prepared of the blend of 10LP10L20-GLL40 and 20LP10L20-GLL40 had a smaller median particle size of 49 μm and a narrow particle size distribution with a CV of 25% (FIG. 9), similar to 120-210052. The apomorphine content of both batches was also substantial (35-37%), representing an EE of approximately 80%. Both formulations had a low residual DCM content and a T_{g} of 46° C.

TABLE 12
Characteristics of apomorphine-loaded microspheres

| | | | | | Residual | | |
|---|---|---|---|---|---|---|---|
| | | PEG | PSD Apomorhine MSP | | DCM | Apomorphine | |
| 120B | | content | D 50 | CV | content | free base load | EE |
| batch | Polymer grade | (mole %) | (μm) | (%) | (ppm) | (wt %) | (%) |
| 210052 | 20LP10L20-GLL40 | 10 | 50 | 30 | 399 | 37.5 | 82.1% |
| 210105 | 10LP10L20-GLL40 | 5 | 71 | 55 | 307 | 37.0 | 81.4% |
| 210132 | 50/50 wt. % blend of | 7.5 | 49 | 25 | <22 | 35.5 | 78.7% |
| | 10LP10L20-GLL40/ | | | | | | |
| | 20LP10L20-GLL40 | | | | | | |

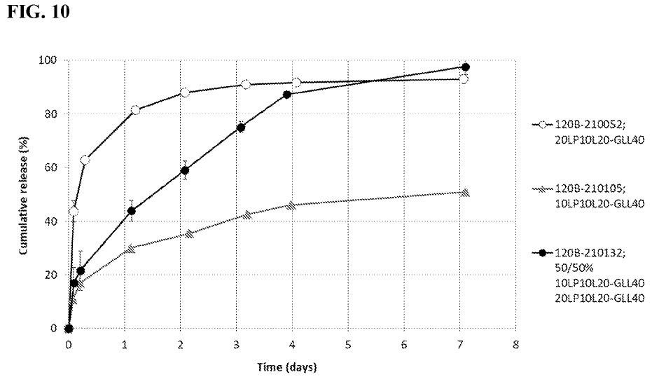

FIG. 10 shows the cumulative in vitro release of apomorphine from the batches that were prepared of 10LP10L20-GLL40 and the 50/50% polymer blend of 10LP10L20-GLL40 and 20LP10L20-GLL40. For reason of comparison, also the release profile of 120B-210052 is shown. As expected, apomorphine was released significantly slower from the less hydrophilic 10LP10L20-GLL40-based apomorphine microspheres with around 50% cumulative release at day 7. Apomorphine microspheres composed of the 50/50% 10LP10L20-GLL40/20LP10L20-GLL40 blend showed intermediate release kinetics with gradual and complete release over a 7-day period and a recovery of around 100%.

What is claimed is:

1. A stable pharmaceutically acceptable formulation comprising:
a microsphere comprising:
a first biodegradable polymer; and
an active drug load of apomorphine or a pharmaceutically acceptable salt thereof.

2. The stable pharmaceutically acceptable formulation of claim 1, wherein the pharmaceutically acceptable salt is an acid addition salt of apomorphine.

3. The stable pharmaceutically acceptable formulation of claim 1 or claim 2, wherein the pharmaceutically acceptable salt is apomorphine hydrochloride.

4. The stable pharmaceutically acceptable formulation of any one of claims 1-3, wherein the microsphere further comprises one or more additional biodegradable polymers.

5. The stable pharmaceutically acceptable formulation of any one of claims 1-4, further comprising one or more antioxidants.

6. The stable pharmaceutically acceptable formulation of claim 5, wherein the one or more antioxidants comprises sodium metabisulfite.

7. The stable pharmaceutically acceptable formulation of claim 5 or claim 6, wherein the one or more antioxidants comprises sodium ascorbate.

8. The stable pharmaceutically acceptable formulation of any one of claims 1-7, wherein the first biodegradable polymer is a copolymer.

9. The stable pharmaceutically acceptable formulation of any one of claims 1-8, wherein the first biodegradable polymer is a multi-block copolymer.

10. The stable pharmaceutically acceptable formulation of claim 9, wherein the multi-block copolymer comprises at least one hydrolysable pre-polymer (A) segment and at least one hydrolysable pre-polymer (B) segment, wherein the segments are linked by a multifunctional chain extender, and wherein the segments are randomly and non-alternatingly distributed over the polymer chain.

11. The stable pharmaceutically acceptable formulation of claim 10, wherein the multi-block copolymer has a T_{g} of about 37° C. or less and a Tₘ of about 110° C. to about 250° C. under physiological conditions, and wherein the pre-polymer (A) segment comprises polyethylene glycol.

12. The stable pharmaceutically acceptable formulation of claim 11, wherein the polyethylene glycol has a Mₙ of about 150 to about 5000 g/mol.

13. The stable pharmaceutically acceptable formulation of claim 10, wherein the multi-block copolymer is amorphous and has a glass transition temperature of 37° C. or less at physiological conditions.

14. The stable pharmaceutically acceptable formulation of any one of claims 10-13, wherein the pre-polymer (A) segment and/or the pre-polymer (B) segment comprises one or more linkages selected from the group consisting of: ester linkages, carbonate linkages, anhydride linkages, ether linkages, and combinations thereof.

15. The stable pharmaceutically acceptable formulation of any one of claims 10-14, wherein the pre-polymer (A) segment comprises one or more polyether groups.

16. The stable pharmaceutically acceptable formulation of claim 15, wherein the one or more polyether groups are selected from the group consisting of: polyethylene glycol, polyethylene glycol-polypropylene glycol, polytetramethylene ether glycol, and combinations thereof.

17. The stable pharmaceutically acceptable formulation of claim 16, wherein said polyether group is polyethylene glycol.

18. The stable pharmaceutically acceptable formulation of any one of claims 10-17, wherein a polyether is present as an additional pre-polymer in the multi-block copolymer.

19. The stable pharmaceutically acceptable formulation of any one of claims 10-18, wherein the pre-polymer (A) segment comprises products of a reaction of at least one cyclic monomer with at least one non-cyclic initiator selected from the group consisting of diols, dicarboxylic acids and hydroxycarboxylic acids.

20. The stable pharmaceutically acceptable formulation of claim 19, wherein the at least one cyclic monomer is selected from the group consisting of glycolide, lactide (D and/or L), ε-caprolactone, δ-valerolactone, trimethylene carbonate, 1,4-dioxane-2-one (para-dioxanone), 1,5-dioxanone-2-one, and a cyclic anhydride.

21. The stable pharmaceutically acceptable formulation of claim 19 or claim 20, wherein the at least one non-cyclic initiator is selected from the group consisting of succinic acid, glutaric acid, adipic acid, sebacic acid, lactic acid, glycolic acid, ethylene glycol, diethylene glycol, 1,4-butanediol, and 1,6-hexanediol.

22. The stable pharmaceutically acceptable formulation of any one of claims 10-21, wherein the pre-polymer (A) segment comprises reactions products of ester forming monomers selected from diols, dicarboxylic acids, and hydroxycarboxylic acids, preferably the pre-polymer (A) segment comprises reaction products of glycolide, lactide (D and/or L), ε-caprolactone, and/or δ-valerolactone.

23. The stable pharmaceutically acceptable formulation of any one of claims 10-22, wherein the content of pre-polymer (A) in the multi-block copolymer is from about 1% to about 90% based on total weight of the multi-block copolymer.

24. The stable pharmaceutically acceptable formulation of any one of claims 10-23, wherein the pre-polymer (A) segment has a Mₙ of about 500 g/mol or more.

25. The stable pharmaceutically acceptable formulation of any one of claims 10-24, wherein the pre-polymer (B) segment comprises a polymer derived from hydroxyalkanoate, glycolide lactide (D and/or L), ε-caprolactone, δ-valerolactone, trimethylene carbonate, 1,4-dioxane-2-one or combinations thereof.

26. The stable pharmaceutically acceptable formulation of any one of claims 10-25, wherein the pre-polymer (B) segment comprises poly(glycolide-co-L lactide).

27. The stable pharmaceutically acceptable formulation of any one of claims 10-26, wherein the pre-polymer (B) segment comprises poly(glycolide-co-L lactide) with a Mₙ of about 1000 g/mol or more.

28. The stable pharmaceutically acceptable formulation of claim 27, wherein the pre-polymer (B) segment comprises poly(glycolide-co-L lactide) with a Mₙ of about 4000 g/mol.

29. The stable pharmaceutically acceptable formulation of any one of claims 26-28, wherein the pre-polymer (B) segment comprises a molar amount of about 5% to about 25% of glycolide relative to combined molar amount of glycolide and L-lactide.

30. The stable pharmaceutically acceptable formulation of claim 29, wherein the pre-polymer (B) segment comprises a molar amount of about 15% of glycolide relative to the combined molar amount of glycolide and L-lactide.

31. The stable pharmaceutically acceptable formulation of any one of claims 10-30, wherein the multi-block copolymer comprises from about 10% to about 99% of the pre-polymer (B) segment relative to the total weight of the multi-block copolymer.

32. The stable pharmaceutically acceptable formulation of any one of claims 10-31, wherein the multifunctional chain extender is a difunctional aliphatic chain extender.

33. The stable pharmaceutically acceptable formulation of claim 32, wherein the difunctional aliphatic chain extender is a diisocyanate.

34. The stable pharmaceutically acceptable formulation of claim 33, wherein the diisocyanate is 1,4-butane diisocyanate.

35. The stable pharmaceutically acceptable formulation of any one of claims 1-9, wherein the first biodegradable polymer is selected from the group consisting of: a polylactide, a polyglycolide, and a poly(lactide-co-glycolide) copolymer.

36. The stable pharmaceutically acceptable formulation of any one of claims 1-9, wherein the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

37. The stable pharmaceutically acceptable formulation of any one of claims 1-36, wherein the first biodegradable polymer is substantially enclosed by a second biodegradable polymer.

38. The stable pharmaceutically acceptable formulation of claim 37, wherein the first biodegradable polymer is not substantially identical in composition to the second biodegradable polymer.

39. The stable pharmaceutically acceptable formulation of any one of claims 1-38, wherein the active drug load of apomorphine or a pharmaceutically acceptable salt thereof in the microsphere is about 30% to about 70% w/w.

40. The stable pharmaceutically acceptable formulation of any one of claims 1-39, wherein the active drug load of apomorphine or a pharmaceutically acceptable salt thereof in the microsphere is about 50% to about 60% w/w.

41. The stable pharmaceutically acceptable formulation of any one of claims 1-39, wherein the active drug load of apomorphine or a pharmaceutically acceptable salt thereof in the microsphere is about 40% to about 50% w/w.

42. The stable pharmaceutically acceptable formulation of any one of claims 1-37, wherein the active drug load of apomorphine or a pharmaceutically acceptable salt thereof in the microsphere is about 30% to about 40% w/w.

43. The stable pharmaceutically acceptable formulation of any one of claims 1-37, wherein the active drug load of apomorphine or a pharmaceutically acceptable salt thereof in the microsphere is about 40% w/w.

44. The stable pharmaceutically acceptable formulation of any one of claims 1-37, wherein the active drug load of apomorphine or a pharmaceutically acceptable salt thereof in the microsphere is about 50% w/w.

45. The stable pharmaceutically acceptable formulation of any one of claims 1-44, wherein the stable pharmaceutically acceptable formulation is substantially sterile.

46. The stable pharmaceutically acceptable formulation of any one of claims 1-45, wherein the stable pharmaceutically acceptable formulation comprises less than 5 wt % of an apomorphine-derived impurity after sealed storage for 24 months at a temperature of 25° C.

47. The stable pharmaceutically acceptable formulation of any one of claims 1-46, wherein the stable pharmaceutically acceptable formulation comprises less than 2% wt/wt of an apomorphine-derived impurity after sealed storage for 24 months at a temperature of 25° C.

48. The stable pharmaceutically acceptable formulation of any one of claims 1-47, wherein the stable pharmaceutically acceptable formulation comprises less than 1% wt/wt of an apomorphine-derived impurity after sealed storage for 24 months at a temperature of 25° C.

49. The stable pharmaceutically acceptable formulation of any one of claims 1-48, wherein the stable pharmaceutically acceptable formulation is substantially free of an apomorphine-derived impurity after sealed storage for 24 months at a temperature of 25° C.

50. The stable pharmaceutically acceptable formulation any one of claims 46-49, wherein the apomorphine-derived impurity is an apomorphine oxidation product.

51. The stable pharmaceutically acceptable formulation of any one of claims 1-50, wherein the stable pharmaceutically acceptable formulation has a shelf life of about 14 days at 25° C. following refrigeration.

52. The stable pharmaceutically acceptable formulation of any one of claims 1-51, wherein the stable pharmaceutically acceptable formulation has a shelf life of about 24 months at 25° C.

53. The stable pharmaceutically acceptable formulation of any one of claims 1-52, wherein the stable pharmaceutically acceptable formulation is stored in a sterile container of about 3.0 mL capacity.

54. The stable pharmaceutically acceptable formulation of any one of claims 1-53, wherein the stable pharmaceutically acceptable formulation is provided in an injector.

55. The stable pharmaceutically acceptable formulation of claim 54, wherein the injector is a disposable pen injector.

56. The stable pharmaceutically acceptable formulation of any one of claims 1-55, wherein the stable pharmaceutically acceptable formulation is suitable for parenteral administration.

57. The stable pharmaceutically acceptable formulation of claim 56, wherein the stable pharmaceutically acceptable formulation is suitable for subcutaneous or intramuscular administration.

58. The stable pharmaceutically acceptable formulation of any one of claims 1-57, wherein the amount of apomorphine or a pharmaceutically acceptable salt thereof per dose is about 40 mg to about 200 mg.

59. The stable pharmaceutically acceptable formulation of any one of claims 1-58, wherein the amount of apomorphine or a pharmaceutically acceptable salt thereof per dose is about 80 mg to about 100 mg.

60. A method of producing a stable pharmaceutically acceptable formulation comprising a microsphere, the method comprising:
providing a first phase comprising:
a first biodegradable polymer; and
an active drug load of apomorphine or a pharmaceutically acceptable salt thereof;
adding a second phase comprising an aqueous surfactant continuously into the first phase to form an emulsion;
adding a quench solution to the emulsion to produce a volume comprising a microsphere; and
washing, filtering, and drying the microsphere to reduce solvent content.

61. A method of producing a stable pharmaceutically acceptable formulation comprising a microsphere, the method comprising:
providing a first phase comprising:
a first biodegradable polymer;
an active drug load of apomorphine or a pharmaceutically acceptable salt thereof; and
a solvent system suitable to dissolve the polymer and apomorphine;
emulsifying the first phase with a second phase, thereby forming an emulsion;
wherein the second phase comprises an aqueous solution which comprises a surfactant; and
removing a substantial portion of the solvent system from the emulsion, thereby obtaining a microsphere.

62. The method of claim 61, further comprising collecting and drying the microspheres.

63. The method of claim 61 or claim 62, wherein the step of emulsifying the first phase with the second phase comprises membrane emulsification using a membrane through which the first phase is introduced into the second phase.

64. The method of any one of claims 61-63, wherein the step of removing the substantial portion of the solvent system from the emulsion comprises extraction of the solvent system by the aqueous solution, wherein the aqueous solution comprises a surfactant.

65. The method of claim 64, wherein the step of removing the substantial portion of the solvent system from the emulsion by extraction is followed by evaporation of the solvent system.

66. The method of any one of claims 61-65, further comprising washing and/or filtering the microspheres.

67. The method of any one of claims 61-66, further comprising drying the microspheres.

68. The method of claim 67, wherein the step of drying comprises one or more of lyophilization, vacuum-drying, and freeze-vacuum drying.

69. The method of any one of claims 60-68, wherein the pharmaceutically acceptable salt is an acid addition salt of apomorphine.

70. The method of any one of claims 60-69, wherein the pharmaceutically acceptable salt is apomorphine hydrochloride.

71. The method of any one of claims 60-70, wherein the first phase comprises a solvent system in which apomorphine or a pharmaceutically acceptable salt thereof has a solubility of about 25 mg/mL or more.

72. The method of any one of claims 60-71, wherein the first phase comprises one or more solvents selected from the group consisting of: dichloromethane, ethyl acetate, chloroform, methanol, benzyl alcohol, dimethyl formamide, dimethyl sulfoxide, N-methyl pyrrolidone and dimethyl acetamide.

73. The method of any one of claims 60-72, wherein the first phase comprises dichloromethane in combination with one or more additional solvents.

74. The method of claim 73, wherein the one or more additional solvents are selected from the group consisting of: dimethyl formamide, dimethyl sulfoxide, and N-methyl pyrrolidone.

75. The method of any one of claims 60-74, wherein the first phase comprises dichloromethane and dimethyl sulfoxide.

76. The method of claim 75, wherein dimethyl sulfoxide is present in an amount of about 5% to about 50% relative to the combined volume of dimethyl sulfoxide and dichloromethane in the first phase.

77. The method of any one of claims 60-76, wherein the first phase comprises about 2% to about 25% by weight of the combined mass of the first biodegradable polymer and the one or more additional biodegradable polymers.

78. The method of any one of claims 60-77, wherein the first phase comprises about 2% to about 25% by weight of apomorphine or a pharmaceutically acceptable salt thereof.

79. The method of any one of claims 60-78, wherein the first phase comprises one or more antioxidants.

80. The method of claim 79, wherein the one or more antioxidants comprises sodium metabisulfite or sodium ascorbate.

81. The method of claim 79 or claim 80, wherein the one or more antioxidants comprises sodium metabisulfite or sodium ascorbate in an amount from about 0.01% to about 5% w/v relative to the first phase.

82. The method of any one of claims 60-81, wherein the second phase comprises polyvinyl alcohol.

83. The method of any one of claims 60-82, wherein the second phase comprises sodium chloride.

84. The method of any one of claims 60-83, wherein the second phase comprises one or more antioxidants.

85. The method of claim 84, wherein the one or more antioxidants comprises sodium metabisulfite.

86. The method of claim 84 or claim 85, wherein the one or more antioxidants comprises sodium metabisulfite in an amount of about 0.1% to about 1% w/v in the second phase.

87. The method of any one of claims 84-86, wherein the one or more antioxidants comprises sodium metabisulfite in an amount of about 0.15% w/v in the second phase.

88. The method of any one of claims 84-87, wherein the one or more antioxidants comprises sodium ascorbate.

89. The method of any one of claims 84-88, wherein the one or more antioxidants comprises sodium ascorbate in an amount of about 0.1% to about 1% w/v in the second phase.

90. The method of any one of claims 84-89, wherein the one or more antioxidants comprises sodium ascorbate in an amount of about 0.15% w/v in the second phase.

91. The method of any one of claims 60-90, wherein the first biodegradable polymer is selected from the group consisting of: a polylactide, a polyglycolide, and a poly(lactide-co-glycolide) copolymer.

92. The method of any one of claims 60-91, wherein the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

93. The method of any one of claims 60-92, wherein the aqueous surfactant comprises one or more of a cationic surfactant, an anionic surfactant, or a non-ionic surfactant.

94. The method of any one of claims 60-93, wherein the second phase further comprises one or more of: a buffer solution, one or more agents for adjusting the viscosity of the aqueous surfactant, and an agent for adjusting the ionic strength of the solution.

95. The method of any one of claims 60-94, wherein the first phase is stirred prior to and/or during the addition of the second phase.

96. The method of any one of claims 60-95, wherein the emulsion is stirred prior to and/or during the addition of the quench solution.

97. The method of any one of claims 60-96, wherein the volume comprising microspheres is stirred prior to and/or during any of the steps of washing, filtering and drying the microspheres.

98. The method of any one of claims 60-97, wherein the first biodegradable polymer is substantially enclosed by a second biodegradable polymer.

99. The method of claim 98, wherein the first biodegradable polymer is not identical in composition to the second biodegradable polymer.

100. The method of any one of claims 60-99, wherein the first phase is prepared by mixing a solution comprising the first biodegradable polymer with a solution comprising the apomorphine or a pharmaceutically acceptable salt thereof.

101. The method of any one of claims 60-100, wherein the first biodegradable polymer and/or the second biodegradable polymer is dissolved in a solvent highly or fully miscible with water selected from the group consisting of: dimethyl sulfoxide, N-methyl-2-pyrrolidone, tetrahydrofuran, tetraglycol, acetone, an acetone/methyl ethyl ketone mixture, an acetone/methyl acetate mixture, a tetrahydrofuran/ethyl acetate mixture, and a tetrahydrofuran/ethyl formate mixture.

102. The method of claim 101, wherein the solvent highly or fully miscible with water is an acetone/methyl ethyl ketone mixture.

103. The method of claim 102, wherein the acetone/methyl ethyl ketone mixture comprises about 70% acetone and about 30% methyl ethyl ketone, by volume.

104. The method of any one of claims 60-103, wherein the first biodegradable polymer and/or the second biodegradable polymer is dissolved in a solvent having limited water solubility selected from the group consisting of: ethyl acetate, methyl acetate, ethyl formate, propyl formate, isopropyl formate, methyl ethyl ketone, and a mixture of two or more thereof.

105. A pre-filled injector comprising:
a stable pharmaceutically acceptable formulation comprising:
a microsphere comprising:
a first biodegradable polymer; and
an active drug load of apomorphine or a pharmaceutically acceptable salt thereof.

106. The pre-filled injector of claim 105, wherein the pharmaceutically acceptable salt is an acid addition salt of apomorphine.

107. The pre-filled injector of claim 105 or claim 106, wherein the pharmaceutically acceptable salt is apomorphine hydrochloride.

108. The pre-filled injector of any one of claims 105-107, wherein the first biodegradable polymer is selected from the group consisting of: a polylactide, a polyglycolide, and a poly(lactide-co-glycolide) copolymer.

109. The pre-filled injector of any one of claims 105-108, wherein the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

110. The pre-filled injector of any one of claims 105-109, wherein the first biodegradable polymer is substantially enclosed by a second biodegradable polymer.

111. The pre-filled injector of claim 110, wherein the first biodegradable polymer is not identical in composition to the second biodegradable polymer.

112. The pre-filled injector of any one of claims 105-111, wherein the active drug load of apomorphine or a pharmaceutically acceptable salt thereof in the microsphere is about 30% to about 70% w/w.

113. The pre-filled injector of any one of claims 105-112, wherein the active drug load of apomorphine or a pharmaceutically acceptable salt thereof in the microsphere is about 50% to about 60% w/w.

114. The pre-filled injector of any one of claims 105-113, wherein the active drug load of apomorphine or a pharmaceutically acceptable salt thereof in the microsphere is about 40% to about 50% w/w.

115. The pre-filled injector of any one of claims 105-114, wherein the active drug load of apomorphine or a pharmaceutically acceptable salt thereof in the microsphere is about 30% to about 40% w/w.

116. The pre-filled injector of any one of claims 105-111, wherein the active drug load of apomorphine or a pharmaceutically acceptable salt thereof in the microsphere is about 40% w/w.

117. The pre-filled injector of any one of claims 105-111, wherein the active drug load of apomorphine or a pharmaceutically acceptable salt thereof in the microsphere is about 45% w/w.

118. The pre-filled injector of any one of claims 105-117, wherein the stable pharmaceutical formulation is substantially sterile.

119. The pre-filled injector of any one of claims 105-118, wherein the stable pharmaceutical formulation has a shelf life of about 14 days at 25° C. following refrigeration.

120. The pre-filled injector of any one of claims 105-119, wherein the stable pharmaceutical formulation has a shelf life of about 24 months at 25° C.

121. The pre-filled injector of any one of claims 105-120, wherein the pre-filled injector is a pen injector or an autoinjector.

122. The pre-filled injector of any one of claims 105-121, wherein the pre-filled injector is disposable.

123. The pre-filled injector of any one of claims 105-122, wherein the pre-filled injector is a disposable pen injector.

124. The pre-filled injector of any one of claims 105-123, wherein the pre-filled injector comprises a cartridge comprising the stable pharmaceutically acceptable formulation.

125. The pre-filled injector of claim 124, wherein the cartridge is a dual chamber cartridge.

126. The pre-filled injector of claim 125, wherein the dual chamber cartridge comprises, in a first chamber, a first volume comprising:
a microsphere comprising an active drug load of apomorphine or a pharmaceutically acceptable salt thereof.

127. The pre-filled injector of claim 125 or claim 126, wherein the dual chamber cartridge comprises, in a second chamber, a second volume comprising:
a dilution medium.

128. The pre-filled injector of claim 127, wherein the first volume and the second volume can be combined to produce the pharmaceutically acceptable formulation of any one of claims 1-59.

129. The pre-filled injector of claim 127 or claim 128, wherein the pre-filled injector is configurable to combine the first volume and the second volume prior to injection.

130. The pre-filled injector of any one of claims 127-129, wherein the pre-filled injector is configurable to combine the first volume and the second volume during injection.

131. The pre-filled injector of any one of claims 127-130, wherein the pre-filled injector is configurable to dispense the first volume and the second volume in about equal amounts by volume.

132. The pre-filled injector of any one of claims 127-131, wherein the pre-filled injector is configurable to dispense the first volume and the second volume in amounts that are not equal by volume.

133. The pre-filled injector of any one of claims 105-132, wherein the pre-filled injector comprises a 18 G to 30 G needle.

134. The pre-filled injector of any one of claims 105-133, wherein the pre-filled injector comprises a 21 G needle.

135. The pre-filled injector of any one of claims 105-134, wherein the amount of the stable pharmaceutically acceptable formulation dispensed in a single injection contains about 40 mg to about 200 mg of apomorphine or a pharmaceutically acceptable salt thereof.

136. The pre-filled injector of any one of claims 105-135, wherein the amount of the stable pharmaceutical formulation dispensed in a single injection contains about 80 mg to about 100 mg of apomorphine or a pharmaceutically acceptable salt thereof.

137. The pre-filled injector of any one of claims 105-136, wherein the amount of the stable pharmaceutical formulation dispensed in a single injection contains about 90 mg of apomorphine or a pharmaceutically acceptable salt thereof.

138. A method of manufacturing a pre-filled injector comprising a stable pharmaceutically acceptable formulation, the method comprising:
preparing a stable pharmaceutically acceptable formulation comprising:
a microsphere comprising:
a first biodegradable polymer; and
an active drug load of apomorphine or a pharmaceutically acceptable salt thereof.
loading a sterile cartridge with the stable pharmaceutically acceptable formulation; and
attaching the sterile cartridge operably to an injector.

139. The method of claim 138, wherein the pharmaceutically acceptable salt is an acid addition salt of apomorphine.

140. The method of claim 138 or claim 139, wherein the pharmaceutically acceptable salt is apomorphine hydrochloride.

141. The method of any one of claims 138-140, wherein the first biodegradable polymer is selected from the group consisting of: a polylactide, a polyglycolide, and a poly(lactide-co-glycolide) copolymer.

142. The method of any one of claims 138-141, wherein the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

143. The method of any one of claims 138-142, wherein the first biodegradable polymer is substantially enclosed by a second biodegradable polymer.

144. The method of claim 142, wherein the first biodegradable polymer is not identical in composition to the second biodegradable polymer.

145. The method of any one of claims 138-144, wherein the active drug load of apomorphine or a pharmaceutically acceptable salt thereof in the microsphere is about 30% to about 70% w/w.

146. The method of any one of claims 138-145, wherein the active drug load of apomorphine or a pharmaceutically acceptable salt thereof in the microsphere is about 50% to about 60% w/w.

147. The method of any one of claims 138-145, wherein the active drug load of apomorphine or a pharmaceutically acceptable salt thereof in the microsphere is about 40% to about 50% w/w.

148. The method of any one of claims 138-145, wherein the active drug load of apomorphine or a pharmaceutically acceptable salt thereof in the microsphere is about 30% to about 40% w/w.

149. The method of any one of claims 138-148, wherein the active drug load of apomorphine or a pharmaceutically acceptable salt thereof in the microsphere is about 40% w/w.

150. The method of any one of claims 138-149, wherein the active drug load of apomorphine or a pharmaceutically acceptable salt thereof in the microsphere is about 45% w/w.

151. The method of any one of claims 138-150, wherein the stable pharmaceutical formulation is substantially sterile.

152. The method of any one of claims 138-151, wherein the stable pharmaceutical formulation has a shelf life of about 14 days at 25° C. following refrigeration.

153. The method of any one of claims 138-152, wherein the stable pharmaceutical formulation has a shelf life of about 24 months at 25° C.

154. The method of any one of claims 138-153, wherein the pre-filled injector is a pen injector or an autoinjector.

155. The method of any one of claims 138-154, wherein the pre-filled injector is disposable.

156. The method of any one of claims 138-155, wherein the pre-filled injector is a disposable pen injector.

157. The method of any one of claims 138-156, wherein the pre-filled injector comprises a cartridge comprising the stable pharmaceutically acceptable formulation.

158. The method of claim 157, wherein the cartridge is a dual chamber cartridge.

159. The method of any one of claims 138-158, wherein the pre-filled injector comprises a 18 G to 30 G needle.

160. The method of any one of claims 138-159, wherein the pre-filled injector comprises a 21 G needle.

161. The method of any one of claims 138-160, wherein the amount of the stable pharmaceutically acceptable formulation dispensed in a single injection contains about 40 mg to about 200 mg of apomorphine or a pharmaceutically acceptable salt thereof.

162. The method of any one of claims 138-161, wherein the amount of the stable pharmaceutically acceptable formulation dispensed in a single injection contains about 80 mg to about 100 mg of apomorphine or a pharmaceutically acceptable salt thereof.

163. The method of any one of claims 138-162, wherein the amount of the stable pharmaceutically acceptable formulation dispensed in a single injection contains about 90 mg of apomorphine or a pharmaceutically acceptable salt thereof.

164. A method of treating motor symptoms associated with Parkinson's disease comprising:
administering to a subject in need thereof a stable pharmaceutically acceptable formulation comprising:
a microsphere comprising:
a first biodegradable polymer; and
an active drug load of apomorphine or a pharmaceutically acceptable salt thereof.

165. The method of claim 164, wherein the pharmaceutically acceptable salt is an acid addition salt of apomorphine.

166. The method of claim 164 or claim 165, wherein the pharmaceutically acceptable salt is apomorphine hydrochloride.

167. The method of any one of claims 164-166, wherein the active drug load of apomorphine or a pharmaceutically acceptable salt thereof in the microsphere is about 30% to about 70% w/w.

168. The method of any one of claims 164-167, wherein the active drug load of apomorphine or a pharmaceutically acceptable salt thereof in the microsphere is about 50% to about 60% w/w.

169. The method of any one of claims 164-168, wherein the active drug load of apomorphine or a pharmaceutically acceptable salt thereof in the microsphere is about 40% to about 50% w/w.

170. The method of any one of claims 164-169, wherein the active drug load of apomorphine or a pharmaceutically acceptable salt thereof in the microsphere is about 30% to about 40% w/w.

171. The method of any one of claims 164-170, wherein the amount of the stable pharmaceutically acceptable formulation administered to the subject contains an amount of apomorphine or a pharmaceutically acceptable salt thereof of about 40 mg to about 200 mg.

172. The method of any one of claims 164-171, wherein the amount of the stable pharmaceutically acceptable formulation administered to the subject contains an amount of apomorphine or a pharmaceutically acceptable salt thereof of about 80 mg to about 100 mg.

173. The method of any one of claims 164-172, wherein the amount of the stable pharmaceutically acceptable formulation administered to the subject contains an amount of apomorphine or a pharmaceutically acceptable salt thereof of about 90 mg.

174. The method of any one of claims 164-173, wherein the stable pharmaceutically acceptable formulation is administered with an injector.

175. The method of any one of claims 164-174, wherein the stable pharmaceutically acceptable formulation is administered parenterally.

176. The method of claim 175, wherein the stable pharmaceutically acceptable formulation is administered subcutaneously or intramuscularly.

177. The method of any one of claims 164-176, wherein administration of the stable pharmaceutically acceptable formulation effects a steady-state plasma concentration of apomorphine following an initial burst of apomorphine in the plasma.

178. The long-acting dosage form of any one of claims 164-177, wherein the steady-state plasma concentration is about 35 μg/L.

179. The long-acting dosage form of any one of claims 164-178, wherein the steady-state plasma concentration is maintained for about 7 days.

180. The method of any one of claims 164-179, wherein the initial burst of apomorphine or a pharmaceutically acceptable salt thereof is about 5% of the total dose of apomorphine or a pharmaceutically acceptable salt thereof.

181. The method of any one of claims 164-180, wherein the initial burst of apomorphine is completed about 0.5 to about 24 hours after injection.

182. The method of any one of claims 164-181, wherein the stable pharmaceutically acceptable formulation is administered once per week.

183. The method of any one of claims 164-182, wherein administration of the stable pharmaceutically acceptable formulation results in less hypotension relative to administration with immediate release subcutaneous apomorphine hydrochloride injection.

184. The method of any one of claims 164-183, wherein administration of the stable pharmaceutically acceptable formulation results in less nausea relative to administration with immediate release subcutaneous apomorphine hydrochloride injection.

185. The method of any one of claims 164-184, wherein administration of the stable pharmaceutically acceptable formulation results in less adverse injection site reactions relative to administration with immediate release subcutaneous apomorphine hydrochloride injection.

186. The method of any one of claims 164-185, wherein the subject in need suffers from Parkinson's disease.

187. The method of claim 186, wherein administration of the stable pharmaceutically acceptable formulation is effective in reducing the occurrence, duration, or severity of intermittent episodes of muscle stiffness and/or loss of muscle control associated with Parkinson's disease.

188. The method of any one of claims 164-187, wherein administration of the stable pharmaceutically acceptable formulation is effective at minimizing hypotension or nausea, or reducing hypotension or nausea by at least 50% when compared to parenteral administration of immediate release apomorphine hydrochloride.

189. The method of any one of claims 164-188, wherein injection site reactions are substantially reduced relative to injection site reactions characteristic of injection with immediate release apomorphine hydrochloride.

190. A long-acting dosage form comprising:
a microsphere comprising:
a first biodegradable polymer; and
about 40 mg to about 200 mg of an active drug load of apomorphine or a pharmaceutically acceptable salt thereof;
wherein administration of a single dose of the long-acting dosage form to a subject results in at least one of the pharmacokinetic parameters selected from the group consisting of:
a steady state plasma profile of apomorphine from day 1 to day 7 following administration exhibiting a mean Cₘₐₓ value no greater than the steady state plasma level of apomorphine provided by 2 mg of immediate release subcutaneous injection of apomorphine hydrochloride;
an apomorphine elimination half-life of about 40 minutes to about 60 minutes; and
a zero-order release profile corresponding to about 12% to about 14% release of the total administered dose of apomorphine or a pharmaceutically acceptable salt thereof per day.

191. The long-acting dosage form of claim 190, wherein the pharmaceutically acceptable salt is an acid addition salt of apomorphine.

192. The long-acting dosage form of claim 190 or claim 191, wherein the pharmaceutically acceptable salt is apomorphine hydrochloride.

193. The long-acting dosage form of any one of claims 190-192, wherein the active drug load of apomorphine or a pharmaceutically acceptable salt thereof in the microsphere is about 30% to about 70% w/w.

194. The long-acting dosage form of any one of claims 190-193, wherein the active drug load of apomorphine or a pharmaceutically acceptable salt thereof in the microsphere is about 50% to about 60% w/w.

195. The long-acting dosage form of any one of claims 190-194, wherein the active drug load of apomorphine or a pharmaceutically acceptable salt thereof in the microsphere is about 40% to about 50% w/w.

196. The long-acting dosage form of any one of claims 190-195, wherein the active drug load of apomorphine or a pharmaceutically acceptable salt thereof in the microsphere is about 30% to about 40% w/w.

197. The long-acting dosage form of any one of claims 190-196, wherein the amount of the stable pharmaceutically acceptable formulation administered to the subject contains an amount of apomorphine or a pharmaceutically acceptable salt thereof of about 80 mg to about 100 mg.

198. The long-acting dosage form of any one of claims 190-197, wherein the amount of the stable pharmaceutically acceptable formulation administered to the subject contains an amount of apomorphine or a pharmaceutically acceptable salt thereof of about 90 mg.

199. The long-acting dosage form of any one of claims 190-198, wherein the first biodegradable polymer is selected from the group consisting of: a polylactide, a polyglycolide, and a poly(lactide-co-glycolide) copolymer.

200. The long-acting dosage form of any one of claims 190-199, wherein the first biodegradable polymer is a poly(lactide-co-glycolide) copolymer, wherein the content of polymerized lactic acid units is at least 50 mol % based on the total amount of polymerized units or blends of poly(lactide-co-glycolide) copolymers with different relative amounts of glycolic acid and lactic acid, wherein the content of polymerized lactic acid units is at least 50 mol % based on the total amount of polymerized units.

201. The long-acting dosage form of any one of claims 190-200, wherein the first biodegradable polymer is substantially enclosed by a second biodegradable polymer.

202. The long-acting dosage form of claim 201, wherein the first biodegradable polymer is not identical in composition to the second biodegradable polymer.

203. The long-acting dosage form of any one of claims 190-202, wherein the active drug load of apomorphine or a pharmaceutically acceptable salt thereof in the microsphere is about 50% w/w.

204. The long-acting dosage form of any one of claims 190-203, wherein the active drug load of apomorphine or a pharmaceutically acceptable salt thereof in the microsphere is about 40% w/w.

205. The long-acting dosage form of any one of claims 190-204, wherein the stable pharmaceutical formulation is substantially sterile.

206. The long-acting dosage form of any one of claims 190-205, wherein the stable pharmaceutical formulation has a shelf life of about 14 days at 25° C. following refrigeration.

207. The long-acting dosage form of any one of claims 190-206, wherein the stable pharmaceutical formulation has a shelf life of about 24 months at 25° C.

208. The long-acting dosage form of any one of claims 190-207, wherein the stable pharmaceutically acceptable formulation is administered from an injector.

209. The long-acting dosage form of claim 208, wherein the injector is a pen injector or an autoinjector.

210. The long-acting dosage form of claim 208 or claim 209, wherein the injector is disposable.

211. The long-acting dosage form of any one of claims 208-210, wherein the injector is a disposable pen injector.

212. The long-acting dosage form of any one of claims 190-211, wherein the injector comprises a cartridge comprising the stable pharmaceutically acceptable formulation.

213. The long-acting dosage form of any one of claims 190-212, wherein the cartridge is a dual chamber cartridge.

214. The long-acting dosage form of any one of claims 190-213, wherein the injector comprises a 18 G to 30 G needle.

215. The long-acting dosage form of any one of claims 190-214, wherein the injector comprises a 21 G needle.

216. The long-acting dosage form of any one of claims 190-215, wherein the injector is a pre-filled injector comprising the stable pharmaceutically acceptable formulation.

217. The long-acting dosage form of any one of claims 190-216, wherein the stable pharmaceutically acceptable formulation is administered parenterally.

218. The long-acting dosage form of claim 217, wherein the stable pharmaceutically acceptable formulation is administered subcutaneously or intramuscularly.

219. The long-acting dosage form of any one of claims 190-218, wherein administration of the stable pharmaceutically acceptable formulation effects a steady-state plasma concentration of apomorphine following an initial burst of apomorphine in the plasma.

220. The long-acting dosage form of any one of claims 190-219, wherein the steady-state plasma concentration is about 35 μg/L.

221. The long-acting dosage form of any one of claims 190-220, wherein the steady-state plasma concentration is maintained for about 7 days.

222. The long-acting dosage form of any one of claims 190-221, wherein the initial burst of apomorphine or a pharmaceutically acceptable salt thereof is about 5% of the total dose of apomorphine or a pharmaceutically acceptable salt thereof.

223. The long-acting dosage form of any one of claims 190-222, wherein following the initial burst of apomorphine or a pharmaceutically acceptable salt thereof, there is an extended release of apomorphine for about 7 days.

224. The long-acting dosage form of any one of claims 190-223, wherein the extended release corresponds to a release of about 5% to about 25% release of the total administered dose of apomorphine or a pharmaceutically acceptable salt thereof per day.

225. The long-acting dosage form of any one of claims 190-224, wherein the extended release corresponds to a release of about 10% to about 15% release of the total administered dose of apomorphine or a pharmaceutically acceptable salt thereof per day.

226. The long-acting dosage form of any one of claims 190-225, wherein following the initial burst of apomorphine or a pharmaceutically acceptable salt thereof, there is a zero-order release profile for about 7 days.

227. The long-acting dosage form of any one of claims 190-226, wherein the zero-order release profile corresponds to a release of about 5% to about 14% release of the total administered dose of apomorphine or a pharmaceutically acceptable salt thereof per day.

228. The long-acting dosage form of any one of claims 190-227, wherein the zero-order release profile corresponds to a release of about 12% to about 14% release of the total administered dose of apomorphine or a pharmaceutically acceptable salt thereof per day.

229. The long-acting dosage form of any one of claims claim 190-228, wherein the elimination half-life of apomorphine is about 40 minutes to about 60 minutes.

230. The long-acting dosage form of any one of claims 190-229, wherein the steady state plasma profile of apomorphine from day 1 to day 7 following administration exhibits a mean Cₘₐₓ value no greater than the steady state plasma level of apomorphine provided by 2 mg of immediate release subcutaneous injection of apomorphine hydrochloride.

231. The long-acting dosage form of any one of claims 190-230, wherein administration of the stable pharmaceutically acceptable formulation results in less hypotension relative to administration with immediate release subcutaneous apomorphine hydrochloride injection.

232. The long-acting dosage form of any one of claims 190-231, wherein administration of the stable pharmaceutically acceptable formulation results in less nausea relative to administration with immediate release subcutaneous apomorphine hydrochloride injection.

233. The long-acting dosage form of any one of claims 190-232, wherein administration of the stable pharmaceutically acceptable formulation results in less adverse injection site reactions relative to administration with immediate release subcutaneous apomorphine hydrochloride injection.

234. A kit comprising:
a first vial comprising a concentrated form of the stable pharmaceutically acceptable formulation of any one of claims 1-59 or of the long-acting dosage form of any one of claims 190-233;
a second vial comprising a pharmaceutically acceptable diluent;
a first syringe suitable for withdrawing the pharmaceutically acceptable diluent from the second vial;
an adapter which can operably attach to the first syringe and is suitable for dispensing the pharmaceutically acceptable diluent into the first vial;
a second syringe suitable for withdrawing a liquid from the second vial and for injecting the liquid into a subject; and
instructions for diluting the concentrated form and for administering the stable pharmaceutically acceptable formulation or the long-acting dosage form to a patient in need thereof.
